(19) United States
(12) Patent Application Publication
(10) Pub. No.: US 2022/0137992 A1
(43) Pub. Date: May 5, 2022

(54) VIRTUAL AGENT TEAM

(71) Applicant: the anti-inflammaging company AG, Zürich (CH)

(72) Inventors: Hans-Lothar Arth, Berlin (DE); Francesco Cavalli, Muzzano (CH); Fabio Cavalli, Muzzano (CH)

(21) Appl. No.: 17/310,980

(22) PCT Filed: Mar. 5, 2020
(86) PCT No.: PCT/EP2020/055949

§ 371 Date: Sep. 2, 2021

(30) Foreign Application Priority Data
Mar. 5, 2019 (EP) 19160914.8

(51) Int. Cl. G06F 9/451; G06F 13/10; G16H 20/30

(52) U.S. Cl. CPC G06F 9/453; G16H 20/30; G06F 13/102

(57) ABSTRACT

The present invention relates to a system for providing a team of at least two virtual agents, the system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest.

The present invention relates to a system for providing a team of at least two virtual agents, the system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest.

### BACKGROUND OF THE INVENTION

Virtual agents in connection with electronic devices, like smartphones or smart speakers are becoming increasingly popular in the modern world and nowadays virtual agents are even indispensable in some cases or applications. Virtual agents may provide an intuitive interface between a user and a system or device in order to enhance and facilitate the interaction therewith. Virtual agents often serve as personal digital assistants in order to organize everyday life, like for example saving appointments in the user's calendar or compiling a shopping list or setting an alarm clock. Moreover providing virtual agents on various wearable electronic devices make them available from all locations and at all times. Therefore one advantage of applying a virtual agent via an electronic device is, for example, that a user may be reminded about an appointment regardless of the location of the user at a predefined point in time. Furthermore, a various number of modern electronic devices are internet-enabled or web-ready, so that virtual agent applications provided on these devices may connect to the World Wide Web and may provide suitable information relating to a user's individual request. A user may therefore decide either to access the World Wide Web through a suitable web browser and to initiate a search request through a search engine or Internet search site by typing in the search request into the appropriate search field or to use a virtual agent application to perform the search. If a search field is provided within the virtual agent application then the user may type in the search request in said search field or optionally if communication through voice recognition is provided the user may just initiate the search request by oral question. In addition, the virtual agent may provide the requested information in oral form to the user. For example a user may communicate through spoken words or a natural language interface with known virtual agents such as Cortana® by Microsoft Corporation or Siri® by Apple Inc. Moreover the virtual agent may be applied hands-free, which is particularly advantageous like, for example, if the user is driving a car and would like to operate a navigation system at the same time.

Virtual agents known in the art are predominantly used to provide specific information, like for example in relation to weather forecast, traffic jams, route guidance and so on or to perform a requested task, like playing a particular audio or video file or setting up a telephone connection. The imitation of natural human speech in connection with oral responses provided by a virtual agent is still a challenging task. As is known human speech is not only characterized by simply speaking out words, moreover human speech is also affected by emotions, mood or the mental state or social surroundings of a speaker and further by body language, like gestures, mimics and so on. Consequently the oral response provided by most of known virtual agents is immediately recognized as artificial voice response and said voice response is characterized in being mainly kind, facial and business-like.

Virtual agents may be provided as embodied agents to provide a visualized virtual agent which is represented graphically with a body. An embodied virtual agent may communicate with a user thereby providing the same verbal and non-verbal cues like a real human being during a conversation. Thus one purpose of applying an embodied agent is to combine gesture, facial expression and speech to enable the imitation of a face-to-face communication with users.

Well-being of an individual or a group is closely linked to certain living conditions of a specific individual or a specific group. A high level in well-being is thereby strongly associated to positive levels relating to specific living conditions. Well-being is for example associated with health, fitness, nutrition, finances, work, job opportunities social status, family status, relationship status, education and/or other living conditions. A high level in well-being is therefore connected with, for example, good health, high education level or by secure financial position. A well-balanced work-life is also associated with a high level in well-being. The term work-life balance describes the balance between time allocated for work and other aspects of life of an individual. Personal interest, family and social or leisure activities for example relate to life domains other than work-life.

In this connection virtual agents may be applied by a user in order to request information relating to the user's individual living condition. A virtual agent may then provide suitable information or may provide a suitable fitness or nutrition plan, which depends on the entered user specific request. Although people may initially be motivated to follow such an individualized training or nutrition plan, it may reveal that such training or nutrition plan may not fit to a person's individual daily routine. For example in a very stressful period it may be difficult to consider and include provided specific health related recommendations during the day. Moreover it is not unlikely that a user may completely forget to request a user-specific health related information or to check the user-specific individual training or nutrition plan. One particular disadvantage of such fitness or nutrition plans lies in the fact that although a user initially might be motivated to follow such strict fitness or nutrition plans after a certain while however motivation to keep on following such scheduled plans decreases drastically over time.

In this regard Sarah Griffiths: “Microsoft Band monitors your fitness levels and sleep quality for $199”, Daily Mail Online, Oct. 30, 2014, p. 1-16 discloses an internet article which describes a device “Microsoft Band” from Microsoft Corporation, which allows users to monitor their fitness and exercise regime, as well as check their texts and e-mails. The wrist-worn device has 10 smart sensors that monitor pulse rate, measure calorie burn and track sleep quality. The device works alongside a health app called “Microsoft Health” that includes a cloud service for users to store and combine health and fitness data. The device has Microsoft's Cortana personal assistant built-in so people can talk to the device to ask it to take notes or set reminders. The smart band from Microsoft aims to give a user a healthier lifestyle by monitoring the user's fitness levels and sleep quality.

The US patent application US 2003/163311 A1 discloses a social agent as an animated computer interface agent with social intelligence. The social intelligence of the agent comes from the ability of the agent to be appealing, affective, adaptive, and appropriate when interacting with the user. The social agent receives an input associated with the user, accesses a user profile associated with the user, extracts context information from the received input and processes the context information and the user profile to produce an adaptive output to be represented by the social agent. The input associated with the user may include physiological data and application program information associated with the user. Extracting context information may include extracting information about an affective state of the user from physiological information, vocal analysis information, or verbal information, a geographical position of a user, information about the application context associated with the user, and linguistic style of the user. An adaptive output to be represented by the social agent may be a verbal expression, a facial expression, or an emotional expression. Aim of this application is to provide a social agent which is appealing, affective, adaptive, and appropriate to the user. Thus, aim is to make the social agent more humanly and to make the dealing with the social agent for the user more convenient.

However, none of the known virtual agents is able to assist a user by providing at the same time to the same topic several opinions, comprising contradictory opinions or recommendations in order to analyse a current situation as complete as possible and giving new ideas how to proceed to the user but leaving the final decision with the user who is due to the contradictory opinions or contradictory recommendations well aware of the possible consequences when following one and not following the contradictory opinion or recommendation.

Moreover, all the commonly known virtual advisors, for instance, for sports, health, food, fashion and the like which run on watches, mobile phones, tablets, eyeglasses and the like are actually used for not longer than in average three weeks as representative surveys have revealed.

This is mostly due to the fact that the users get bored be the virtual advisor and do not want to be patronized or infantilized. This is especially true for the virtual health and sport advisors which give plans how, which and when to do sport or what, when in which amounts to eat.

Thus, it is the objective of the present invention to provide a new generation of virtual advisors or virtual agents which overcome the afore-mentioned drawbacks. It is the objective of the present invention to enable the user to obtain a clear and full picture of a certain situation without guiding the user in one single direction. Thus, it could also be stated that the objective of the present invention is to provide a system and a method and a team of virtual agents is reduced or eliminates stress of the user which the user has in certain situations by the provision of contradictory opinions or contradictory recommendations. Thus, objective of the present invention is to provide a method, system and visualized virtual agents for reduction of stress of the user thereby reducing stress related diseases and disorders of the user like heart attack, stroke and cardiovascular diseases.

This objective is solved by providing a team of virtual agents which are distinctively presented to the user and which give recommendations which could even be contradictory due to the field of interest assigned to the respective virtual agent. Like a president who has a brain trust consisting of several advisors. Consequently, the advice of the finance minister in regard to a certain situation might be contradictory to the advice of the minister of defense or the advice of the foreign minister might go in quite a different direction than the advice of the minister for social affairs or the recommendations of the minister for economic affairs are often the opposite of the recommendations of the minister for the environment. The president and here the user received the different recommendations made under different or even contradictory views of the situation, can make up his mind and finally act as he thinks it is the best. Thus, the methods, system, and team of virtual agents are especially beneficial to the user in situations of stress. Under high stress situations a user can lose to overview quickly and might get into a helpless situation not knowing it he should quit immediately, continue, do nothing, or proceed differently. In such situations stress can be reduced drastically by providing not only one guidance that the user has to follow, but by providing several including contradictory recommendations, so that the user gets a clear picture of the situation and can pick for him the most suitable way to proceed. This leaves the final decision with the user who has the huge benefit to be aware of the advantages and disadvantages of the various options and recommendations how he could proceed.

This also avoids to bore, patronize, or infantilize the user. The user receives the recommendations from at least two virtual agents at the same time to the same current state or situation of the user and the user is free to decide which recommendation he will follow or if he ignores all recommendations or follows both recommendations only in part. Thus, even knowing that the recommendation selected be the user might bear certain disadvantages, it is still up to the user to decide what to do.

Thus, the above objective is solved by providing a system, a method, and team of visualized virtual agents which are distinctively presented to the user and which give bidirectional or multidirectional or preferably contradictory recommendations, but not unidirectional recommendations within a close temporal succession.

Further advantageous embodiments of the invention result from the dependent claims, the description, the figures and the examples.

First acceptability trials performed by the applicant have clearly demonstrated that the team of virtual agents is well accepted by the users and all users continued to use this team of virtual agents until the end of the four months trial period.

### BRIEF DESCRIPTION OF THE INVENTION

Thus, the present invention is directed to a system for providing a team of at least two virtual agents, the system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest.

The terms “virtual agent” and “visualized virtual agents” are used herein as synonyms.

Consequently, when using the term “visualized virtual agents” instead of “virtual agent” the present invention is directed to a system for providing a team of at least two visualized virtual agents, the system comprising at least one central processing unit, at least one non-transitory computer readable storage medium, at least one input device, and at least one output device, wherein the visualized virtual agents are distinctively presented to a user by the at least one output device and each of the at least two visualized virtual agents is assigned to a different field of interest and each of the at least two visualized virtual agents is configured to provide visual and/or audio recommendations of the field of interest assigned to the respective visualized virtual agent to the user on the basis of user-specific data and on the basis of the current state of the user, wherein at least two visualized virtual agents of the team assigned to at least two different fields of interest provide recommendations to the user in the current state so that the user is provided with bidirectional, multidirectional, or even contradictory recommendations of at least two different fields of interest.

Furthermore, it has been found that the system displaying the team of the at least two virtual agents reduces stress of the user in certain situations and preferably occupational stress. This again reduces the risk and the occurrence of cardiac arrhythmia, stroke, heart attack and other cardiovascular diseases and disorders.

Therefore, the present application is directed to a system for stress reduction of a user comprising at least one central processing unit, at least one non-transitory computer readable storage medium, at least one input device, and at least one output device for displaying a team of at least two virtual agents, wherein the virtual agents are distinctively presented to the user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest.

The system described herein displays the team of the at least two virtual agents in real-time or near real-time on the output device.

Thus, the present invention further relates to a system for providing a team of at least two virtual agents in real-time or nearly real-time, the system comprising at least one central processing unit, at least one non-transitory computer readable storage medium, at least one input device, and at least one output device, wherein the virtual agents are configured to be distinctively presented to a user by the at least one output device and each of the at least two virtual agents is configured to be assigned to a different field of interest and each of the at least two virtual agents is configured to provide visual and/or audio recommendations in the field of interest assigned to the respective virtual agent to the user on the basis of user-specific data and on the basis of the current state of the user, wherein at least two virtual agents of the team assigned to at least two different fields of interest are configured to provide recommendations to the user for the current state of the user so that the user is provided with bidirectional, multidirectional, or even contradictory recommendations of at least two different fields of interest.

The team of at least two virtual agents could also be regarded as a network of virtual agents.

Thus, the present invention further relates to a system for providing a virtual agent network, the system comprising at least two virtual agents concurrently running on a system comprising at least one central processing unit, at least one non-transitory computer readable storage medium, at least one input, and at least one output device, wherein each of the at least two virtual agents is configured to provide visual and/or audio responses in form of recommendations to a user on the basis of at least one predefined field of interest and on the basis of user-specific data and a current state of the user, wherein the at least one predefined field of interest is selected from a group of at least two different fields of interest, wherein for each of the at least two virtual agents at least one different field of interest is selected from the group of the at least two different fields of interest so that the user is provided with bidirectional, multidirectional, or even contradictory recommendations of at least two different fields of interest.

In a preferred embodiment each of the at least two virtual agents is configured to provide auto-actively recommendations to the user. In a preferred embodiment the recommendations are further based on activity data of the user and/or physio-psychological data of the user. The recommendations may be further based on a present behavior and/or a current physical condition and/or a current mental state of the user. In a preferred embodiment the physio-psychological data of the user are based on a present behavior/non-verbal behavior and/or a current physiological condition and/or a current mental state/psychological state and/or medical condition of the user. In a preferred embodiment each of the at least two virtual agents is displayed two dimensionally and/or three dimensionally on a display device. In a preferred embodiment the visual response of each of the two virtual agents comprises a posture, and/or motion of each virtual agent and wherein the audio response of each of the two virtual agents comprises a sound, a sound volume, an emphasis, and/or an accent of each of the virtual agents on the basis of the activity data of the user and/or physio-psychological data of the user and/or the assigned field of interest of the respective virtual agent. In a preferred embodiment the posture, and/or the motion of each of the two virtual agents and the sound, sound volume, emphasis and/or accent of each virtual agents depend on the assigned field of interest. In a preferred embodiment at least on sensor is configured to acquire physio-psychological parameters of the user directly by speech recognition, face recognition, measurement of pulse, measurement of breathing, measurement of blood pressure, body temperature, and/or measurement of the electric conductivity of the skin. In a preferred embodiment each virtual agent is in form of a stylized chameleon, wherein the form of the stylized chameleon for each virtual agent depends on the assigned field of interest of the respective virtual agent.

The present invention further relates to a computer-implemented method for provision of a team of at least two virtual agents running on a system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest, the method comprising the following steps: assigning a field of interest to each of the at least two virtual agents, wherein at least two virtual agents are assigned to a different field of interest, collecting and/or acquiring user-specific data, analyzing user-specific data, determining a current state of the user, generating recommendations for each of the at least two virtual agents on the basis of the assigned field of interest of the respective virtual agent, the user-specific data and the current state of the user, presenting and/or providing the generated recommendations to the user by the at least one output-device.

The present invention further relates to a computing device for provision of a team of at least two virtual agents, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest, the computing device comprising at least one processing unit, at least one non-transitory computer readable storage medium storing computer-readable instructions for provision of the team of at least two virtual agent executable by the at least one processing unit, at least on input device for collecting user-specific data, and at least one output device for presenting the recommendations to the user.

The US patent application US 2003/163311 A1 disclosing a social agent as an animated computer interface agent could be regarded as closest prior art. This social agent receives an input associated with the user, accesses the user profile, extracts context information from the received input and processes the context information and the user profile to produce an adaptive output to be represented by the social agent. Objective of this patent application is to make the virtual agent to a social agent which is appealing, affective, adaptive, and appropriate to the user.

However, it is not disclosed how this social agent could reduce stress situations of the user by providing bidirectional or multidirectional and/or contradictory recommendations of different fields to the user so that the stress situation of the user is analyzed for the user and a clear picture of the situation is provided to the user, leaving the final decision how to proceed with the user without dictating to the user what he shall do.

### DESCRIPTION OF THE INVENTION

The present invention is directed to a system for providing a team of at least two virtual agents, the system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional or multidirectional and/or contradictory recommendations of at least two different fields of interest.

In addition, the present invention is directed to a system for stress reduction of a user comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional or multidirectional and/or contradictory recommendations of at least two different fields of interest.

The bidirectional or multidirectional and/or contradictory recommendations have to be provided in close temporal succession, i.e. the recommendations should be provided one after another within a short time frame, i.e. within seconds to up to a view minutes and preferably within less than 60 seconds.

The term “bidirectional” refer to recommendations which are not of similar type and which are not interchangeable like the recommendations to go biking for an hour or to go skiing for an hour or the recommendations to eat potatoes with chicken or noodles with beef or the recommendations to wear jeans together with a leather jacket or corduroy trousers with a pullover.

Rather this term refer to recommendations which are made in light of different fields of interest and are not of similar type and are not interchangeable, because each recommendation has advantages and disadvantages over the other recommendation(s). Moreover, the recommendations overlap only in part and are slightly antithetic up to being completely contradictory.

The term “contradictory” refers to recommendations which are the opposite of each other like donate all your money or donate nothing.

The term “multidirectional” refers to three or more recommendations which are not of similar type and which are not interchangeable. Thus, these are recommendations which are made under three or more different views and which consequently go into three or more different directions.

The following example will illustrate how the team of virtual agents works and how it provides recommendations. One can imagine that a married couple had founded a small enterprise twenty years ago and succeeded to build it up over the years to a medium size enterprise with about 100 employees. Now being close to 60 years old, the couple decided to sell the medium size enterprise and commenced negotiations with a big company interested in buying the enterprise. Today is another day of negotiations which are close to finalization. After further hours of exhausting negotiations during a short brake, the virtual agent team makes the following recommendations to the current state. The health agent recommends not to proceed with the negotiations for at least two days, because on the basis of the physiological condition like heart rate, blood pressure, pulse rate, skin/body temperature and the like of the wife obtained through a watch worn by the wife, she has due to the enormous stress a drastically increased risk to have an epileptic seizure. Due to the health data of the wife stored in the system, the team of virtual agents is able to make such an assessment, because in the past she had sporadic epileptic seizure when the stress exceeded a certain level. The virtual health agent recommends delaying the negotiations for at least two days, because health is more important than making a good deal and in the worst case the wife could die during an epileptic seizure.

Immediately thereafter, the virtual social agent provides the option that the negotiations could be continued by only the husband while the wife could take a rest. Thereby the negotiations could be continued while the wife would decrease the risk of an epileptic seizure or would prevent an epileptic seizure.

Also in close temporal succession, the virtual financial agent recommended that the negotiations should be continued after this short brake by the married couple and should be finalized soon, because in the internet a new press release was published that the negotiation partner and potential buyer of the medium size enterprise will soon start negotiations to buy a company which is a direct competitor of the medium size enterprise of the married couple. Thus, not taking the risk that the negotiation partner will in the future buy the competitor company, the couple should continue and try to finalize the deal, since the conditions of the deal are also quite good. Moreover, the virtual financial agent reminded that during the last 20 years the married couple had made all important decision together and that also in the present situation it might be good to decide together in order to avoid future problems concerning the interpersonal relationship of the couple.

Thus, the team of virtual agents provides multidirectional recommendations, wherein two of which are contradictory, namely stopping the negotiations and continuing the negotiations by the married couple. The third recommendation is in a third direction, namely not continuing the negotiations by the wife and continuing the negotiations by her husband. Thus, here we have multidirectional or three-directional recommendations.

However, the important aspect of this new generation of virtual agents is that a team of virtual agents provides recommendations under different views, because each virtual agent which is distinctively presented to the user has its own field of interest and provides at least one recommendation on the basis of his respective field of interest like in the above example the financial field, the health field and the social/partnership field between the married couple. But most important is still that the user which is the married couple in the above example makes the final decision and the team of virtual agents does not dictate an advice, it rather analyzes the current state or the current situation and provides recommendations under different views of the current state, thereby providing a full picture of the situation to the user and probably provides new ideas to the user, but leaves the final decision with the user and does not dictate to the user the “best” way to proceed, since no system is able to access which the “best” way actually is. Providing this analysis of the current state or current situation of the user decreases the stress the user has at the current situation, preserves the user's health and in addition supports the user to find for him the best way to proceed.

Thus, in regard to the above example, the wife might comment that she feels over-stressed but would still like to close the deal especially in light of this new press release. Thus, the couple may decide that although they made all important decisions during the last 20 years together, she will quit for today but her husband should continue so that the deal could be closed today.

Consequently, the system, method, and team of virtual agents disclosed herein reduces or eliminates the stress of the user to take the right decision in a certain situation. If a user is over a longer period of time such as several hours under a situation of stress, the user might get headless which further increases the stress on the user. The present invention is able to reduce this stress by analyzing the current situation of the user and providing recommendations which give a complete or almost complete picture of the current situation of the user without domineering the user how to decide and how to proceed.

Virtual agents are commonly applied in order to provide information in response to a user's specific request or task. Providing information is based on a communication of facts which have been requested by a user. Additionally, a virtual agent may also play a role as a virtual agent, which provides recommendations or advices relating to a specific topic or field of interest. For example, a virtual agent may take over a role as a mentor or a guide or motivation trainer. With regard to a specific topic or specific field of interest or in connection with a user's condition a virtual agent may provide individual recommendations or advices, which depend, for example, on user's individual preferences and/or user's emotions or mood and/or on a specific field of interest and/or a current state of the user and/or on other suitable user-specific data. In general recommendations or advices have a non-binding character, but in conjunction with these recommendations or advices activities of a user may be positively influenced. This is particularly advantageous over virtual agents known in the art, which are merely configured to provide a strict scheduled plan, like nutritional or physical training plan to a user, since in cases a user fails to follow the provided scheduled plan, the frustration of the user increases, which may even cause the user to completely dismiss the scheduled plan. Due to the non-binding character of the recommendations or advices the frustration of the user may be minimized, particularly in times the user is prevented of considering the provided recommendations or advices. Virtual agents or other related systems and methods known in the art for provision of a specific scheduled plan to a user are often merely connected to one living condition of user or to one specific field of interest. For example virtual agents or related systems and methods for provision of merely a fitness and nutrition plan to a user are well known in the art. Such virtual agents or system and methods are directed to assist a user to reach usually a predefined goal to increase fitness or a physical condition of a user commonly by fitness tracking in connection with several fitness tracker devices. It often happens that users set too high predefined goals, which may drastically increase the risk of frustration of a user. Furthermore, it often turns out to be difficult to incorporate such fitness and nutritional plans into everyday life of a user. Surprisingly it has been found that is particularly advantageous to provide a virtual agent or a team of at least two virtual agents which provide recommendations to the user with regard to a plurality of living conditions of a user or a plurality of fields of interest, particularly wherein the recommendations to the user are provided from different perspective view of specific fields of interest to the user to improve well-being of a user. Surprisingly it has been found that such a team of at least two virtual agents, wherein each of the at least two virtual agents is assigned to a different field of interest and wherein each of the two virtual agents is configured to provide recommendations to a user in the field of interest assigned to the respective virtual agent is advantageous over virtual agents and related systems and methods known in the art to improve well-being of a user.

The term “virtual agent” as described herein generally relates to computer readable instructions that may be provided with and executed on an electronic device, comprising a memory and one or more processor, such as a personal computer or such as a wearable electronic device like a mobile phone e.g. a smart phone or like a smart watch. The team of the at least two virtual agents of the present invention may be implemented with various electronic devices or may be configured to connect to various electronic devices. The at least two virtual agents or the system may comprise common functionalities of virtual agents known in the art like may be configured to answer questions or to provide requested information. The at least two virtual agents or the system may be configured to communicate with a user. The at least two virtual agents or the system may be configured to obtain interaction context of a user, like characteristics of a user's speech, the identity of a user, the expression and gestures of a user. The at least two virtual agents may be configured to provide gestures, facial expressions and speech of each of the at least two virtual agents to enable the imitation of a face-to-face communication with users. The at least two virtual agents may be configured to express emotions or mood of each virtual agent. The at least two virtual agents may be configured to provide visual changes and/or verbal instructions of each virtual agent, thus the at least two virtual agents may be configured to provide visual and/or audible behavioral responses to a user. The at least two virtual agents or the system may be configured to observe, analyze and respond to user's requests or to observe, analyze and respond to monitored user-specific parameter. Each of the at least two virtual agents may be a guide, like a health guide, a personal coach, a personal agent, a personal assistant, a personal trainer, an advisor, like a health advisor, and/or a personal companion of a user. The at least two virtual agents or the system may be configured to connect to a network and/or to a server, like a client server or a cloud server. The at least two virtual agents or the system may be configured to access one or more databases. The at least two virtual agents or the system may be configured to provide information or requested data, which may be obtained from one or more databases. The at least two virtual agents or the system may be configured to access a third party service.

The term “field of interest” as described herein generally relates to a specific topic or specific living condition of a user. A field of interest may be associated with health, fitness, nutrition, financial situation, work, job opportunities, job situation, social status, family status, relationship status, education, emotional status, ambient situation, hygienic conditions, availability of medicinal products and medical care, hobbies, travelling, housing conditions, insurances, retirement, social and financial security, mobility, social status, material possessions, property, luxury needs, ethnic, cultural, linguistic and religious identity, sexuality, self-discovery, personal wishes and dreams, legal protection, international security, economic development, social progress and/or other living conditions.

A high level in well-being is strongly associated to positive levels related to specific living conditions or fields of interest. Happiness is a mental or emotional state of well-being. The thresholds between the different fields of interest cannot be strictly defined, since a plurality of factors or parameters which have a specific impact on one specific field of interest may also have an impact on another field of interest. A positive impact on a specific field of interest may however have a negative impact on another field of interest. A reasonable balance between the different fields of interest is required to ensure or to lead to a high level in well-being. In case of an individual it is often required that a weighting factor is applied to the different fields of interest since the fields of interest which are important for well-being of a specific individual may depend on the age of the individual, family and cultural background, personal interests and hobbies, and personality and so on. An example wherein a positive impact on one field of interest leads to a positive impact on another field of interest may relate to a positive progress in fitness of an individual which may have additionally a positive impact on health or physical condition in general. A positive impact on one field of interest like hobbies, travelling, luxury needs and material possession is often accompanied with cash dispensing. Cash dispensing may have a negative impact on the financial situation of an individual. From the perspective of an independent observer of the financial situation of an individual it may be concluded that well-being of said individual decreases since the financial situation has worsened. However cash dispensing with regard to hobbies, travelling, luxury needs and material possession may have a positive influence on well-being, which is also well respected in the modern society. Thus, from a perspective of another independent observer one would conclude that well-being of said individual has reached a higher level particularly due to the financial investments. The aforementioned examples particularly show that the impact of specific factors or parameter on well-being strongly depend on the perspective view.

In order to reach a high level in well-being it may not be suitable to follow a specific scheduled plan with regard to one or more specific fields of interest. For example, a user which may particularly be interested in saving money and may follow a scheduled financial plan thereby following strict instructions not to spend money for travelling or for any material possessions. Also in cases a user would like to follow a strict fitness plan and/or nutritional plan the time to do anything else may be restricted due to a strict scheduled daily routine. Additionally a nutritional plan may include instructions not to eat food which the user in general would like to eat. However such strict scheduled plans may be reasonable, for example, if the user has a high debt burden or has massive overweight etc. Nevertheless well-being in general depends on so many parameters and factors that it is quiet impossible to provide a specific plan to satisfy all of the living conditions or fields of interest. Moreover for many of the fields of interest like family status or relationship status a scheduled plan including specific instructions may even not be practicable.

Virtual agents known in the art which are configured to provide assistance in connection to merely one specific field of interest may be regarded as an observer providing guidance merely from one specific point of view. In contrary virtual agents configured to provide information or to perform specific task in response to a request of a user are generally lacking such a perspective view. As described above in connection with specific circumstances of the current situation or current state of a user it may be particularly reasonable to provide guidance or recommendations to a user from different perspectives or different points of view. It was surprisingly found that provision of recommendations to a user from different perspectives provides beneficial and improved guidance to a user. It could be shown that a team of at least two virtual agents assigned to different fields of interest configured to provide distinctively recommendations to a user from different points of view provides improved guidance to user to improve well-being of a user. Furthermore it could be shown that it is particularly beneficial that the provided recommendations are not provided in form of a scheduled plan, since recommendations from different perspectives may provide even contradictory recommendations. With regard to the above described example a virtual agent allocated to the field of interest of financial situation would provide recommendations to not to spend money for example for travelling and for luxury needs. However a virtual agent allocated to the field of interest of travelling or luxury needs would recommend to make a journey or to buy a specific luxury product.

The team of at least two virtual agents may be configured to provide visual and/or audio recommendations to a user inter alia on the basis of a field of interest of the respective virtual agent. In one embodiment the selection of the field of interest to be assigned to each of the at least two virtual agents may be defined by the user or may be determined automatically on the basis of user-specific data provided by the user and/or stored in one or more databases. Determining a suitable field of interest to be assigned to the at least two virtual agents may depend on a current state of a user or user's interest and/or goals. However in a preferred embodiment the fields of interest are not selected entirely to correspond with the interest of a user as a whole. The fields of interest preferably are selected from distinct topics or living conditions of a user. The more the selected fields of interest differ from each other the more the user may be provided with bidirectional, multidirectional, or even contradictory recommendations by the at least two virtual agents. Preferably selection of the field of interest to be assigned to the at least two virtual agents in general should particularly be relevant to living circumstances of the user. In one preferred embodiment the user may initially select a specific number of provided virtual agents and the fields of interest to be assigned to the respective virtual agents. In one preferred embodiment two fields of interest may be only selected together to enable provision of bidirectional or multidirectional recommendations or even contradictory recommendations to a user. For example the user may select initially that one of the virtual agents of the team of at least two virtual agent should be assigned to the field of interest of financial situation, the team of at least two virtual agents may configured to define that a second virtual agent of the team of the at least two virtual agents must be assigned to the field of interest of hobbies, luxury need or material possession. In order to determine and/or to generate visual and/or audio recommendations to a user inter alia on the basis of a field of interest assigned to the respective virtual agent, data related to each of the fields of interest may be stored in one or more database. These data may further include suitable information how a living condition or the well-being of a user may be improved in connection to the respective field of interest. The data related to the fields of interest preferably include neutral data and/or informative data related to the respective field of interest. Thus, the data related to the fields of interest may include predefined rules how well-being of a user may be improved to the respective field of interest.

Thus, the present invention relates to a system for providing a team of at least two virtual agents running preferably in real time on the system comprising at least one central processing unit, at least one non-transitory computer readable storage medium, at least one input device, and at least one output device, wherein the virtual agents are distinctively or separately or individually or independently presented to a user by the at least one output device and each of the at least two virtual agents is assigned or connected or allocated to a different field of interest and each of the at least two virtual agents is configured to provide visual and/or audio recommendations to the user in the field of interest assigned or connected or allocated to the respective virtual agent on the basis of user-specific data and on the basis of the current state of the user, wherein at least two virtual agents of the team assigned or connected or allocated to at least two different fields of interest provide recommendations to the user for the current state of the user so that the user is provided with bidirectional, multidirectional, or even contradictory recommendations of at least two different fields of interest.

Thus, the present invention relates to a system for providing a team of at least two virtual agents running preferably in real time on a system comprising at least one central processing unit, at least one non-transitory computer readable storage medium, at least one input device, and at least one output device, wherein the virtual agents are distinctively or separately or individually or independently presented to a user by the at least one output device and each of the at least two virtual agents is assigned or connected or allocated to a different field of interest and each of the at least two virtual agents is configured to provide visual and/or audio recommendations to the user in the field of interest assigned or connected or allocated to the respective virtual agent on the basis of user-specific data and on the basis of the current state of the user, wherein at least two virtual agents of the team assigned or connected or allocated to at least two different fields of interest provide recommendations to the user for the current state of the user so that the user is provided with bidirectional, multidirectional, or even contradictory recommendations of at least two different fields of interest, wherein the field of interest is selected from a group consisting of health, fitness, nutrition, financial situation, work, job opportunities, job situation, social status, family status, relationship status, education, emotional status, ambient situation, hygienic conditions, availability of medicinal products and medical care, hobbies, travelling, housing conditions, insurances, retirement, social and financial security, mobility, social status, material possessions, property, luxury needs, ethnic, cultural, linguistic and religious identity, sexuality, self-discovery, personal wishes and dreams, legal protection, international security, economic development and/or social progress.

The present invention further relates to a system for providing a team of at least two virtual agents running preferably in real time on a system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest selected from a group consisting of health, fitness, nutrition, financial situation, work, job opportunities, job situation, social status, family status, relationship status, education, emotional status, ambient situation, hygienic conditions, availability of medicinal products and medical care, hobbies, travelling, housing conditions, insurances, retirement, social and financial security, mobility, social status, material possessions, property, luxury needs, ethnic, cultural, linguistic and religious identity, sexuality, self-discovery, personal wishes and dreams, legal protection, international security, economic development, social progress and/or other living conditions and each of the at least two virtual agents is configured to provide visual and/or audio recommendations to the user in the field of interest assigned to the respective virtual agent on the basis of user-specific data and on the basis of the current state of the user, wherein at least two virtual agents of the team assigned to at least two different fields of interest provide recommendations to the user for the current state of the user so that the user is provided with bidirectional, multidirectional, or even contradictory recommendations of at least two different fields of interest.

In order to provide individualized recommendations to a user by the at least two virtual agents assigned to different fields of interest, the recommendations may be further based on user-specific data and a current state of a user to allow provision of visual and audio recommendations to a user in the field of interest assigned to the respective virtual agent on the basis of user-specific data and on the basis of the current state of the user, wherein at least two virtual agents of the team assigned to at least two different fields of interest provide recommendations to the user for the current state of the user so that the user is provided with bidirectional, multidirectional, or even contradictory recommendations of at least two different fields of interest. The user-specific data may comprise user-specific data of health, fitness, nutrition, financial situation, work, job opportunities, job situation, social status, family status, relationship status, education, emotional status, ambient situation, hygienic conditions, availability of medicinal products and medical care, hobbies, travelling, housing conditions, insurances, retirement, social and financial security, mobility, social status, material possessions, property, luxury needs, ethnic, cultural, linguistic and religious identity, sexuality, self-discovery, personal wishes and dreams, legal protection, international security, economic development, social progress and/or other living conditions of a user. The current state of the user may be determined on the basis of user-specific data of health, fitness, nutrition, financial situation, work, job opportunities, job situation, social status, family status, relationship status, education, emotional status, ambient situation, hygienic conditions, availability of medicinal products and medical care, hobbies, travelling, housing conditions, insurances, retirement, social and financial security, mobility, social status, material possessions, property, luxury needs, ethnic, cultural, linguistic and religious identity, sexuality, self-discovery, personal wishes and dreams, legal protection, international security, economic development, social progress and/or other living conditions of the user. The user-specific data for the fields of interest including health, fitness, nutrition, financial situation, work, job opportunities, job situation, social status, family status, relationship status, education, emotional status, ambient situation, hygienic conditions, availability of medicinal products and medical care, hobbies, travelling, housing conditions, insurances, retirement, social and financial security, mobility, social status, material possessions, property, luxury needs, ethnic, cultural, linguistic and religious identity, sexuality, self-discovery, personal wishes and dreams, legal protection, international security, economic development, social progress and/or other living conditions of a user may be stored in one or more databases accessible by the team of at least two virtual agents and may be provided directly by the user or may be collected from different sources for example other applications running on an electronic device of the user or databases of other programs for which the team of the at least two virtual agent has permission to access and receive said user-specific data. As non-limiting examples health data of a user may be stored in a user-specific health file which may include medical history data of a user, diseases, allergies, physical impairments, financial data may include bank data, bank account data, bank saving accounts, investment funds, credits, debts, financial assets and other assets, job situation data may include curriculum vitae, former jobs, job qualifications and the like, family status may include information about family members, marital status, number of children, relatives and the like, relationship status data may include information of friends, friendships, partners, partnership and the like. The user-specific data in connection to the different fields of interest may be stored in one or more database. The user-specific data in connection to the different fields of interest may be stored separately for each of the field of interest in a respective database. The team of at least two virtual agents may be configured to access the one or more databases wherein the user-specific data to the different fields of interest are stored. Each of the at least two virtual agents may be configured to access the user-specific data of the field of interest assigned to the respective virtual agent. The one or more databases comprising user-specific data in connection to the different fields of interest may be updated over time, in real-time or in near real-time.

The recommendations generated by the team of at least two virtual agents may further depend on user-specific parameter and on the current state of a user, for example, activity data of a user and/or physio-psychological data of user. Thus, the at least two virtual agents may provide recommendations to a user further based on activity data of a user and/or physio-psychological data of a user. Preferably, the recommendations generated by the at least two virtual agents may be based on at least one user-specific parameter, wherein the user-specific data of the at least one user-specific parameter may be acquired directly from a sensor, for example, by acquiring activity data of a user using at least one sensor and/or acquiring physio-psychological data of user using at least one sensor. The team of the at least two virtual agents of the present invention may be configured to monitor user-specific parameter related to the different fields of interest. Thus, the team of the at least two virtual agents of the present invention may be configured to monitor user-specific parameter, like activity data of a user and/or physio-psychological data of a user over time, in real-time or in near real-time. The at least two virtual agents of the present invention may be configured to acquire user-specific data for at least one user-specific parameter of activity data of a user and/or physio-psychological data of a user directly from at least one input device, like at least one sensor. Thus, the at least two virtual agents of the present invention may be configured to acquire user-specific data for at least one user-specific parameter relating to activity data of a user and/or physio-psychological data of a user using a sensor over time, in real-time or in near real-time. On the basis of the monitored and/or the acquired activity data of user and/or the monitored and/or the acquired physio-psychological data of a user the at least two virtual agents may be configured to generate recommendations to a user. Thus, the at least two virtual agents may be further configured to analyze, to determine and to generate recommendations to a user on the basis of monitored and/or acquired activity data of a user and/or monitored and/or acquired physio-psychological data of a user.

The at least two virtual agents may be further configured to determine a current state of the user. The at least two virtual agents may be further configured to initially determine a current state of the user in order to determine an initial selection of suitable user-specific parameter, which may be particularly suitable to serve as a basis for generating recommendations to a user. Thus, it may be advantageous if the at least two virtual agents may be further configured to initially determine a current state of the user in order to determine an initial selection of one or more sensors which may be used to directly acquire user-specific data for one or more user-specific parameter, which may be particularly suitable to serve as a basis for generating recommendations to a user.

The current state of the user may be determined based on user-specific parameter, like activity data of a user and/or physio-psychological data of a user and/or suitable user-specific data of health, fitness, nutrition, financial situation, work, job opportunities, job situation, social status, family status, relationship status, education, emotional status, ambient situation, hygienic conditions, availability of medicinal products and medical care, hobbies, travelling, housing conditions, insurances, retirement, social and financial security, mobility, social status, material possessions, property, luxury needs, ethnic, cultural, linguistic and religious identity, sexuality, self-discovery, personal wishes and dreams, legal protection, international security, economic development, social progress and/or other living conditions of the user. With other words the at least two virtual agents may be configured to monitor, to collect and to analyze a large amount of data for a plurality of different user-specific parameter, preferably by acquiring said data by using at least one input device, for example a sensor, to obtain for example the activity data of a user and/or physio-psychological data of a user and be further configured to determine an initial selection of one or more suitable user-specific parameter selected from the entire user-specific parameter in order to determine a specific selection of one or more user-specific parameter, which may serve the basis for generating recommendations to a user.

The at least two virtual agents may be further configured to determine a current state of a user or an overall state of a user based on activity data of a user and/or physio-psychological data of a user or user-specific data of health, fitness, nutrition, financial situation, work, job opportunities, job situation, social status, family status, relationship status, education, emotional status, ambient situation, hygienic conditions, availability of medicinal products and medical care, hobbies, travelling, housing conditions, insurances, retirement, social and financial security, mobility, social status, material possessions, property, luxury needs, ethnic, cultural, linguistic and religious identity, sexuality, self-discovery, personal wishes and dreams, legal protection, international security, economic development, social progress and/or other living conditions of the user. The at least two virtual agent may be configured to provide recommendations on the basis of a current state of a user. The current state of a user may be determined by acquiring user-specific data for at least on user-specific parameter, the user-specific parameter may comprise activity data of a user and/or physio-psychological data of a user, by using at least one input device, like for example at least one sensor.

The “current state of the user” as described herein further relates to activity data of a user or physio-psychological data of a user like to the mood or emotions of a user or like to specific activities of the user like physical training or eating or relaxing and the like. Thus, the at least two virtual agent may be configured to provide recommendations to a user on the basis of a current behavior and/or the current physiological state and/or the current mental state and/or a current medical state of the user. Thus, the at least two virtual agent may be configured to provide recommendations to a user on the basis of a current behavior and/or the current physiological state and/or the current mental state and/or a current medical state of the user and on the basis of the field of interest assigned to the respective virtual agent. In one embodiment the at least two virtual agents may be configured to provide recommendations on the basis of inter alia a current state of a user which may be determined on the basis of activity data of a user. In another embodiment the at least two virtual agents may be configured to provide recommendations to the user on the basis of inter alia a current state of a user which may be determined on the basis of physio-psychological data of a user. In another embodiment the current state of a user may be determined on the basis of activity data of a user and physio-psychological data of a user. Activity data of a user may include current activities of a user like for example what the user is doing, like sleeping, eating, doing physical exercises etc. For example, when a user is running, the current state of a user may refer to a running state of the user. For example, when the at least two virtual agent have determined a running state of a user, for times the activity data of the user indicate that the user is running, wherein the activity data may be acquired by using a sensor like a GPS tracker or step counting sensor, the at least two virtual agent may be further configured to acquire the user-specific data from, for example, the GPS tracker or step counting sensor and may be therefore configured to monitor and to automatically determine if the user stops running. The at least two virtual agent may be further configured to acquire physio-psychological data from the user from one or more sensors during the activity of the user. For example, in connection with the speed, the duration, the heart rate etc. The at least two virtual agents may be configured to automatically recognize, thus to determine if one of these physio-psychological data changes, like for example if the user slows down, or if the heart rate increases or decreases.

As an example a current state of a user may relate to times the user is running. Thus, a running state of a user may refer to a period of time when the user is running. Another example of a current state of a user may refer to times when the user is relaxing on his couch. Thus, a relaxing state of a user may refer to a period of time when the user is relaxing on his coach. Thus, the at least two virtual agents may be configured to determine a current state of a user which may relate to a running state of a user or a relaxing state of a user. The at least two virtual agent may be further configured to provide different recommendations to a user for different states of a user, thus for different current states of a user. For example, the at least two virtual agent may be configured to provide different recommendations to a user for a running state of a user then for a relaxing state of user. Thus, the at least two virtual agent may be configured to monitor and to analyze activity data of a user and/or the physio-psychological data of a user over time, in real-time or in near-real time and be further configured to determine a state of a user, preferably a current state of a user based on the monitored activity data of a user and/or physio-psychological data of a user. After the current state of the user has been determined by the at least two virtual agent, the at least two virtual agent may be further configured to continue to monitor and to analyze activity data of a user and/or physio-psychological data of a user over time, in real time or in near-real time and be further configured to automatically recognize and thus to determine a change of the current state of the user based on the monitored and/or acquired and analyzed activity data of a user and/or physio-psychological data of a user and/or based on monitored user-specific data of the different fields of interest. Thus, after determining a first state of a user the at least two virtual agents may be further configured to determine a second state of a user based on a monitored change of activity data of a user and/or physio-psychological data of a user. The at least two virtual agents may be configured to provide recommendations to a user based on a first selection of one or more user-specific parameter of activity data of a user and/or physio-psychological data of a user for a first state of a user and be further configured to provide recommendations to a user based on a second selection of one or more user-specific parameter of activity data of a user and/or physio-psychological data of a user for a second state of a user. The at least two virtual agents may be further configured to change between the first state of the user and the second state of a user. Preferably the at least two virtual agent may be configured to determine at least one state of the user. Preferably the at least two virtual agents may be configured to determine a current state of a user over time, in real time or near real time.

The at least two virtual agent may be configured to provide equal or different recommendations to a user for different states of a user. Thus, the at least two virtual agents may be configured to provide recommendations for different states of a user either based on the same selection of one or more user-specific parameter of the activity data of a user and/or physiological data of a user or may be based on different selections of one or more user-specific parameter of the activity data of a user and/or physio-psychological data of a user. For example, the at least two virtual agents may be configured to provide recommendations to a user based on a lower number of user-specific parameter for a relaxing state of a user then for a running state of a user. The at least two virtual agent may be configured to provide equal or different recommendations to a user for different states of a user and for different fields of interest assigned to the respective virtual agent. Thus, each of the at least two virtual agent may be configured to provide visual and/or audio recommendations to a user on the basis of a field of interest assigned to the respective virtual agent and on the basis of user-specific data and a current state of the user. Each of the at least two virtual agent may provide visual and/or audio recommendations on the basis of the same user-specific data and the current state of a user, however as at least two of the at least two virtual agent are assigned to different fields of interest, the user may be provided with bidirectional, multidirectional, or even contradictory recommendations at least due to the assignment to different fields of interest and thus due to the different perspective views of the at least two virtual agents.

Thus, in one embodiment of the present invention the system for providing the at least two virtual agents may be configured to provide recommendations to a user for at least one state of a user, preferably a current state of a user, wherein the at least two virtual agents may be configured to provide a visual and/or audio recommendations to the user, wherein the at least one state of the user, preferably the current state of the user may be determined on the basis of activity data of the user and/or physio-psychological data of a user and wherein the at least two virtual agent may be further configured to adapt the recommendations to monitored changes of activity data of the user and/or physio-psychological data of the user and thus to monitored changes of the current state of the user, wherein each of the at least two virtual agents is assigned to a different field of interest and each of the at least two virtual agents is configured to provide visual and/or audio recommendations to the user of the field of interest assigned to the respective virtual agent and on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest.

The generation of recommendations to a user may also depend on the currently used electronic device of a user. With other word the currently used electronic device of a user where the team of at least two virtual agent providing recommendations to a user may be stored and executed by one or more processors. With other word the currently used electronic device of a user where the computer readable instructions for generating of a team of at least two virtual agents providing recommendations to a user may be executed by at least one processor. Different electronic devices may comprise different computing systems, and not every system may be particular suitable for rendering or generating of the at least two virtual agents providing visual recommendations to a user by applying the entire data input relating to user-specific parameter like activity data of a user and/or physio-psychological data of a user or user-specific data of different fields of interest. For example, the at least two virtual agents may be implemented and executed on a watch, like a smart watch, comprising a display to enable presentation of the at least two virtual agents. The at least two virtual agents may be rendered and presented on the display over time and the at least two virtual agents may be configured to generate recommendations over time, in real time or in real time to a user.

Furthermore, a specific electronic device may include integral input devices, e.g. sensors. The at least two virtual agents may be configured to provide recommendations to a user on the basis of activity data of the user and/or physio-psychological data of the user acquired by using at least one sensor of the electronic device. The at least two virtual agents may be configured to generate the recommendations to a user without requesting other activity data of the user and/or physio-psychological data of the user. Thus, it may not be required that the at least two virtual agents may access, for example, one or more databases on one or more server and/or client server and/or secured server. Moreover, the activity data of a user and/or physio-psychological data of a user in order to determine a current state of a user may be monitored and analyzed with a specific electronic device. In case the at least two virtual agents determine a change of the current state of the user, the electronic device may then transmit the determined change of the current state to another electronic device used by the user. The other electronic device may then adapt the recommendations to the determined change of the current state of the user and may then be configured to generate the recommendations to the user on the basis of other activity data and/or physio-psychological data of the user. Thus, it is not required that said other electronic device independently monitors in real time or near-real time all activity data of the user and/or all physio-psychological data of the user over time, in real time or near-real time to determine a current state of a user.

The activity data of a user and/or the physio-psychological data of user may be monitored by using at least one input device, e.g. at least one sensor. Thus, the at least two virtual agents may be connected to at least one input device, e.g. at least one sensor. The at least two virtual agent may be configured to receive generated output signals from at least one input device. The at least two virtual agents may be configured to receive output signals from at least one sensor. The at least two virtual agent may be configured to receive the generated output signals from one or more sensors. The at least two virtual agents may be configured to receive generated output signals from one or more input devices. The at least one input device and/or at least one sensor may be an integral part of an electronic device where the team of at least two virtual agents may be executed or the at least one input device and/or at least one sensor may be located externally to such an electronic device. The at least one input device, e.g. at least one sensor may be configured to receive input signals related to a user for recognizing and/or measuring and/or monitoring an activity parameter of a user and/or physio-psychologic parameter of a user and be further configured to generate output data from the captured input signals related to the user. The at least two virtual agents may be connected to at least one input device and/or at least one sensor to monitor activity data of a user and/or physio-psychological data of a user. The at least two virtual agents may be configured to analyze the activity data of a user and/or physio-psychological data of a user. Thus, the at least two virtual agents may be configured to determine a current state of a user on the basis of the monitored activity data of a user and/or physio-psychological data of a user, which may be monitored by using at least one input device, e.g. at least one sensor. The at least one input device and/or at least one sensor may be configured to transmit the generated output data to the at least two virtual agent, for example, user-specific data related to verbal and non-verbal behavior of the user. The at least one input device, e.g. a sensor may include suitable audio-visual sensors, activity sensors, physiological sensors, biometric sensors and/or other sensors. The at least one sensor may be configured to transmit (wired or wirelessly) the output data directly to a processor. The at least one input device and/or at least one sensor may be directly attached to the user. Alternatively the at least one input device and/or at least one sensor may be disposed within an electronic device and/or another device utilized by the user.

The virtual agent and/or the system may be configured to receive generated output signals from the one or more input devices, e.g. one or more sensors to obtain and receive activity data of a user and/or physio-psychological data of a user as input data related to the user. Thus, the at least two virtual agents and/or the system may be configured to receive input data relating to activity data of a user and/or physio-psychological data of a user from one or more input devices, e.g. one or more sensors. Preferably the at least two virtual agents and/or the system may be configured to acquire input data relating to activity data of a user and/or physio-psychological data of a user directly from at least one sensor. The input data may include one or more of behavior data of the user, physiological data of the user, psychological data of the user, medical data of the user and/or other information or data related to the user. The at least two virtual agents and/or the system may receive the output signals generated by at least one input device and/or at least one sensor within or outside the computing system. The at least two virtual agents and/or the system may be configured to receive user-specific input data from sensors, and/or other resources by electronically querying and/or requesting said data from such devices and receiving the activity data of the user and/or physio-psychological data of the user in response.

The at least two virtual agents and/or one or more processors of the system may be configured to receive activity data of a user and/or physio-psychological data of a user and further be configured to acquire activity data of a user and/or physio-psychological data of the user directly from at least one sensor, and/or may be configured to receive activity data of a user and/or physio-psychological data of a user in any way that allows the team of at least two virtual agents or the computing system for generating the team of at least two virtual agents to function as described herein.

For example, physiological data of a user, which may relate to a current physical and/or physiological condition of a user may include heart rate, blood pressure, weight, pulse rate, blood chemistry, blood oxygen saturation, blood glucose level, hydration information, respiration rate, breathing information, skin/body temperature, conductivity of the skin, brain activity, physical movements and/or lack of movement, user specific activity data and/or physiological data may further include data related to performance and/or non-performance of daily activities, activity duration information, physical pain information, and/or other physiological data. Examples of behavior data of a user may include the users' demeanor, voice, look, gestures, manners, attitude, vocal expression, verbal expression, and/or other behavior data. Examples of user-specific psychological data may include user's personality, mood, emotions, perceptions, cognitions, and/or other psychological data related to the user. The at least two virtual agents may be configured to extract user-specific data from acquired input signals transmitted by at least one input device or sensor for example via automatic speech recognition and/or audio-visual behavior recognition. The at least two virtual agent may be configured to extract user-specific input data from audio-visual input (e.g. user voice and/or video received from a microphone, and/or camera). Automatic speech recognition may include identifying words and phrases in the user's speech and converting them into machine readable format. Audio-visual behavior recognition may include facial recognition, body language recognition, recognition of acoustic non-content properties of speech (rhythm, emphasis, intonation, pitch, intensity, rate, etc.) and/or other behavior.

The at least two virtual agents may be further configured to receive and/or to acquire input data related to other users or one or more users. For example users in the same age group, same gender, users with similarities in their physiological, behavior, psychological, and/or medical information, and/or other users with other similarities to the user.

Non-verbal communication is characterized by visual cues such as body language, distance of communicators and physical environments and appearance and also of voice and of touch. Non-verbal communication can also include the use of time and eye contact and the actions of looking while talking and listening, frequency of glances, patterns of fixation, pupil dilation, and blink rate and the like. Human speech contains also non-verbal elements, including voice quality, rate, pitch, volume and speaking style, as well as prosodic features such as rhythm, intonation, and stress. Non-verbal communication also depends on environmental conditions where communication takes place, on physical characteristics of communicators, and on behaviors of communicators during interaction. Non-verbal communication is characterized by encoding and decoding processes of non-verbal cues. Encoding is related to the provision of information in form of facial expression, gestures, and postures, whereby decoding is related to the interpretation or understanding of said provided information. Some non-verbal cues relate to the inherent human behavior, for example, like smiling, crying, or laughing. Non-verbal communication may involve non-verbal cues in form of gestures. Gestures may be performed with hands, arms or body, and also include movements of the head, face and eyes. Gestures can also be categorized as either speech-independent or speech-related. Speech-independent gestures are dependent upon culturally accepted interpretation and have a direct verbal translation. Speech-related gestures are used in parallel with verbal speech. This form of non-verbal communication is used to emphasize the message that is being communicated. Speech-related gestures are intended to provide supplemental information to a verbal message such as pointing to an object of discussion. Facial expressions serve as a practical means of communication. With all the various muscles that precisely control mouth, lips, eyes, nose, forehead, and jaw, human faces are estimated to be capable of more than ten thousand different expressions. In addition many emotions, including happiness, sadness, anger, fear, surprise, disgust, shame, anguish and interest are universally recognized. Display of emotions can generally be categorized into two groups: negative and positive. Negative emotions usually manifest as increased tension in various muscle groups: Tightening of jaw muscles, furrowing of forehead, squinting eyes, or lip occlusion. In contrast, positive emotions are revealed by the loosening of the furrowed lines on the forehead, relaxation of the muscles around the mouth, and widening of the eye area. Some hand movements are not considered to be gestures like scratching, fidgeting, rubbing or tapping. These hand movements may serve as the basis for dispositional inferences of the user's emotion (nervous, uncomfortable, bored). Eye contact is the primary non-verbal way of indicating engagement, interest, attention and involvement. Disinterest is highly noticeable when little or no eye contact is made in a social setting. When an individual is interested, however, the pupils will dilate. In addition non-verbal cues can consist of physiological aspects including pulse rate as well as levels of perspiration. Eye contact and facial expressions provide important social and emotional information.

The at least two virtual agent may be configured to receive user-specific data with regard to one or more user-specific parameter based on activity data of a user and/or physio-psychologic data of a user from one or more input devices like one or more sensors. The at least two virtual agent may be configured to acquire user-specific data for the one or more user-specific parameter of activity data of a user and/or physio-psychologic data of a user directly from at least one sensor. The one or more input devices or one or more sensors may be wired or wirelessly connected to the at least two virtual agent. Therefore the virtual agent may be connected to at least one input device and/or at least one sensor for recognizing and/or measuring physio-psychologic parameters of the user by speech recognition, face recognition, measurement of pulse, measurement of breathing, measurement of blood pressure and/or measurement of the electric conductivity of the skin. The one or more sensors may be configured to transmit user-specific data for one or more user-specific parameter for activity data of user and/or physio-psychological data of a user to the visualized virtual agent. The at least two virtual agents may be further configured to store the transmitted user-specific data of activity data of a user and/or physio-psychological data of a user in a user-specific database and/or a memory and/or a storage device. The at least two virtual agent may be configured to receive user-specific data as input data of activity data of a user and/or physio-psychological data from one or more input devices like one or more sensors and may be further configured to store the transmitted input data of activity data of a user and/or physio-psychological data of a user in a memory and/or a storage device and/or a server.

The at least two virtual agents may be further configured to generate a user-specific database wherein the transmitted input data of the activity data of the user and/or physio-psychological data of the user may be stored. On the basis of the transmitted input data relating to activity data of a user and/or physio-psychological data of a user from one or more input devices like the one or more sensors, the visualized virtual agent may be configured to provide recommendations to the user based on these transmitted user-specific data of activity data of a user and/or physio-psychological data of a user and/or stored data of activity data of a user and/or physio-psychological data of a user. For example in case the virtual agent acquires user-specific data for at least one user-specific parameter which may relate to activity data of a user and/or physio-psychological data of a user from a sensor which may be configured to monitor the pulse rate of a user, the at least two virtual agent may be configured to generate recommendations to the user on the basis of said user-specific data. After generating the recommendations on the basis of the transmitted user-specific data of activity data of a user and/or physio-psychological data of a user from the input device like the one or more sensors, the at least two virtual agents may be further configured to cause and render presentation of the generated recommendations on a display device. The at least two virtual agents may be further configured to cause and render presentation of the generated recommendations on several display devices. Therefore, the at least two virtual agents and/or the system may be configured to generate recommendations on one or more display devices.

Examples of input devices are sensors comprising or consisting of a heart rate sensor, a blood pressure sensor/monitor, a weight scale, motion sensors, an optical sensor, a video sensor, an audio sensor, a blood glucose monitor, a blood oxygen saturation monitor, a hydration monitor, a skin/body temperature thermometer, a respiration monitor, electroencephalogram (EEG) electrodes, bed sensors, accelerometer, activity sensors/trackers, and/or other sensors, a video camera e.g. web cam, a depth sensor, electro dermal activity (EDA) sensor, portable global positioning system (GPS) sensor that tracks the location of the user over time, in real time or in near real time. The sensors may be configured to generate any output signals related to input data relating to activity data of a user and/or physio-psychological data of a user that allows the virtual agent or computing system for generating a team of at least two virtual agents to function as described herein. The one or more sensors may be disposed in a plurality of locations within or outside of the computing system. For example the one or more sensors may be attached to the user, coupled with the user interface, located in a medical device used by the user, positioned to point at the user like a video camera, and/or in other locations within or outside of the system. The one or more sensors may be configured to capture facial expressions of the user, location of the user, posture of the user, voice of the user, electro-dermal activity of the user etc. The at least two virtual agents and/or the system may be configured to determine values indicative of valence, arousal, and engagement of the user, based on the input data of activity data of a user and/or physio-psychological data of a user acquired from the one or one or more sensors, which may be monitored over time or in real time or near real time. Input data relating to activity data of a user and/or physio-psychological data of a user from the one or more sensors may be transmitted directly or indirectly to a central server or a local server. Input data relating to activity data of a user and/or physio-psychological data of a user from the one or more sensors may be transmitted directly or indirectly in addition to or instead to an electronic device.

The at least two virtual agent and/or the system may be connected to at least one input device, e.g. one or more sensors, wherein the one or more sensors are configured to transmit input data of activity data of a user and/or physio-psychological data of a user to the at least two virtual agents. The at least two virtual agent may be configured to provide recommendations on the basis of said transmitted input data of activity data of a user and/or physio-psychological data of a user. For example the at least two virtual agents may be connected to one or more video capturing sensors like one or more cameras. The one or more cameras may be integral parts of several different electronic devices of the user (e.g. smartphone, tablet, PDAs, TV) or be located in other devices of the user (e.g. refrigerator, weighing machine, and the like) or be positioned at specific positions in a room (e.g. living room, bedroom and/or kitchen) in a user's home. The at least two virtual agents may be further connected to one or more audio sensors like one or more microphones. The one or more microphones may be an integral part of several different electronic devices of the user (e.g. smartphone, tablet, PDAs, TV) or be located in other devices of the user (e.g. refrigerator, weighing machine, and the like) or be positioned at specific positions in a room (e.g. living room, bedroom and/or kitchen) in a user's home. The at least two virtual agents may be further connected to a portable global positioning system (GPS) sensor, which may be configured to transmit tracking data of the user's position to the at least two virtual agents. The at least two virtual agents may be further connected to other suitable input devices and/or sensors. For example, the at least two virtual agents may be configured to receive data related to the facial expression of a user, which may be transmitted by the one or more sensors, e.g. the one or more cameras to the at least two virtual agents. The at least two virtual agents and/or the system may then provide recommendations on the basis of the monitored facial expressions of the user. The at least two virtual agents may be further configured to receive input data of the facial expressions of a user from one or more sensors over time, e.g. from the one or more cameras. The team of the at least two virtual agents and/or the system of the present invention is therefore further configured to generate output signals in form of recommendations and be further configured to transmit said output signals to one or more output devices, e.g. one or more display devices and/or one or more microphones.

The team of at least two virtual agents and/or the system may be configured to provide visual and/or audio recommendations to a user on the basis of an assigned field of interest of the respective virtual agent, user-specific data and a current state of the user. Thus, the recommendations provided by the at least two virtual agents are preferably not predefined recommendations. The user may be provided with different types of recommendations for example substitutive or compensative recommendations to improve guidance or assistance to reach a specific goal or items comparison or topic related facts and information to provide specific information with regard to the assigned field of interest of the respective virtual agent. In one preferred embodiment the at least two virtual agents and/or the system may receive data transmitted through an application programming interface (API), for example, through representational state transfer application (REST) API services. In one embodiment the content which may be transmitted may be provided by keywords for the respective recommendation which may be provided to the electronic device of the user. The at least two virtual agents and/or the system may be configured to elaborate a sentence on the basis of said keywords or may be configured to present data files, like images or videos and the like. The at least two virtual agents may be configured to determine a sentence with said keywords. The at least two virtual agents and/or the system may be configured to receive data transmitted through API or may be configured to transmit a response or recommendation via API. In one embodiment of the invention the team of at least two virtual agents is provided in one application. Thus, preferably the team of at least two virtual agents do not relate to provision of several independent applications or independent computer readable instructions, wherein each of the at least two virtual agents may be provided by several independent applications to the user.

The team of at least two virtual agents and/or the system may be further configured to provide auto-actively visual and/or audio recommendations to a user. The term “auto-active” as described herein relates to the provision of recommendations to the user on the basis of an assigned field of interest of the respective virtual agent, of user-specific data and the current state of the user by the at least two virtual agents irrespective of whether the user has initiated a specific request or whether the user stays in active correspondence with the at least two virtual agents. Thus, the team of at least two virtual agents may be configured to provide auto-actively visual and/or audio recommendations to a user if a triggering event or a trigger is met. A trigger or triggering event for provision of auto-active recommendations may include monitored, analysed and determined changes of user-specific data of the fields of interest, user-specific data and/or current state of a user. The trigger or triggering event may be further based on activity data of a user or physio-psychological data of a user. The trigger or triggering event for provision of auto-active visual and/or audio recommendations to a user may further include a previous response or a previous recommendation of one of the at least two virtual agents. Thus, after or shortly after one of the at least two virtual agents has provided a recommendation to the user, for example the content of the recommendation may met a triggering event to provide a recommendation to a user by a second virtual agent. Preferably the second virtual agent configured to provide a recommendation to a user in reaction to a previously provided recommendation of a first virtual agent may be assigned to a different field of interest than said first virtual agent. The recommendation of the second virtual agent may further fulfil the triggering event for provision of recommendations to the user by the first virtual agent. The trigger or triggering event for provision of auto-active visual and/or audio recommendations to a user may further include a reaction or response of the user to a previously provided response or a previously provided recommendation of one of the at least two virtual agents. Thus, after or shortly after one of the at least two virtual agents has provided a recommendation to the user, the reaction or response of the user with regard to said recommendation may fulfil a triggering event to provide a recommendation to a user by a second virtual agent. Preferably the second virtual agent configured to provide a recommendation to a user in reaction to a previously provided recommendation of a first virtual agent may be assigned to a different field of interest than said first virtual agent. The reaction or response of the user to the recommendation of the second virtual agent may further fulfil the triggering event for provision of recommendations to the user by the first virtual agent. Thus, in a preferred embodiment the communication of the team of the at least two virtual agents with the user may be compared with a real conversation between at least three discussion participants. The user may be provided with bidirectional, multidirectional, or even contradictory recommendations by the at least two virtual agents assigned to different fields of interest on which basis the user may be provided with improved assistance or guidance to decide which of the recommendations he would like to follow. Since each of the at least virtual agents may be configured to provide visual and/or audio recommendations to the user on the basis of a field of interest assigned to the respective virtual agent and user-specific data and the current state of the user in response to a previously provided recommendation(s) of another of the at least two virtual agent assigned to a different field of interest and/or in response to the reaction or response of the user to such a previously provided recommendation(s), such a conversation between the team of the at least two virtual agent with the user may provide improved assistance or guidance to a user to find the optimal path for any further action. Furthermore, since the at least two virtual agents may provide bidirectional, multidirectional, or even contradictory recommendations to the user, one of the bidirectional, multidirectional, or contradictory recommendations may comprise a recommendation to the user which the user had not thought about itself which may further provide improved guidance or assistance to the user particularly in order to improve well-being of the user, since said bidirectional, multidirectional, or even contradictory recommendation may comprise content, which the user would not have taken into consideration without provision of said recommendations by the at least two virtual agents.

The present invention further relates to a system for providing a team of at least two virtual agents, the system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest, wherein each of the at least two virtual agents is configured to provide the recommendations auto-actively to the user.

In one preferred embodiment the team of at least two virtual agents may comprise two virtual agents assigned to two different fields of interest. In another preferred embodiment the team of at least two virtual agents may comprise three virtual agents, wherein at least two of the three virtual agents are assigned to two different fields of interest, or wherein the three of the virtual agents are assigned to three different fields of interest. In another preferred embodiment the team of at least two virtual agents may comprise four or five or six virtual agents. Thus, the team of at least two virtual agents may comprise 2 to 20 virtual agents, preferably 2 to 15 virtual agents, more preferably 2 to 10 virtual agents or preferably 2-5 virtual agents and particularly preferred 2 to 4 virtual agents. In another preferred embodiments the team of at least two virtual agents may comprise 3 to 10 virtual agents, preferably 3 to 5 virtual agents. In another preferred embodiment the team of at least two virtual agents may comprise 4 to 8 virtual agents, preferably 4 to 6 virtual agents. In a preferred embodiment each of the at least two virtual agents may be assigned to different fields of interest.

The at least two virtual agents of the present invention may be rendered and presented on a display device. The at least two virtual agents of the present invention may be rendered and presented distinctively on a display device. Thus, each of the at least two virtual agents may be rendered and presented on a display device to give the user the impression that he communicates with different or independent virtual agents at the same time. The at least two virtual agents may be rendered and presented to the user at the same time on the display device, wherein each of the at least virtual agents may be configured to provide visual and/or audio recommendations to the user on the basis of a field of interest assigned to the respective virtual agent, user-specific data and a current state of the user. The at least two virtual agents may be rendered and presented to the user consecutively on the display device, wherein each of the at least virtual agents may be configured to provide visual and/or audio recommendations to the user on the basis of a field of interest assigned to the respective virtual agent, user-specific data and a current state of the user. In an embodiment wherein the team of at least two virtual agents may comprise three or more virtual agents at least two of the virtual agents may be rendered and presented to the user at the same time or consecutively on the display device, wherein each of the at least virtual agents may be configured to provide visual and/or audio recommendations to the user on the basis of a field of interest assigned to the respective virtual agent, user-specific data and a current state of the user.

The at least two virtual agents may provide visual responses combined with audio responses not solely in written form or spoken words, furthermore by specific virtual visual appearances of each of the at least two virtual agents. Thus the at least two virtual agent may be represented graphically with a visual virtual body and may be configured to interact with a user in verbal and non-verbal manners. In this connection interactions with a user in verbal manners may relate to audio responses in form of oral responses provided by the at least two virtual agents and interactions with a user in non-verbal manners may relate to visual responses in form of body language responses provided by the at least two virtual agents. The at least two virtual agents comprising, for example, means of speech recognition and non-verbal behavior recognition may be configured to respond to verbal and non-verbal communications from the user. For example, the at least two virtual agents may be configured to respond in non-verbal manners characterized by showing expressions or gestures on the basis of the present behavior and/or the current physical condition and/or the current state of the user. The at least two virtual agents may further comprise additional means for recognizing specific conditions of a user, like for example means for recognizing an emotional state of a user in order to provide emotional oral responses and emotional body language responses to a user. Therefore, the at least two virtual agents may be configured to provide expressions or gestures adapted to the user's emotional state, emotions or mood. Therefore the at least two virtual agents may be configured to provide simulated emotions.

In order to provide a visual virtual appearance of each of the at least two virtual agents to the user a suitable display device may be connected to the at least two virtual agents, which may be configured to cause presentation of each of the at least two virtual agents on said display device. The display device may be configured to provide a two dimensionally or a three dimensionally visual representation of each of the at least two virtual agents. Therefore each of the at least two virtual agent may be displayed two dimensionally or three dimensionally by the display device. The display device may comprise for example a graphical user interface, a display, a touchscreen, and/or other devices. The display device may include monitors, mobile communication devices, user information systems, and/or other graphic or electronic displays. The display device may be configured to receive generated visual signals and to render and cause visualization of each of the at least two virtual agents on the display device. The display may additionally be configured to render and present each of the at least two virtual agents together with other information, for example information of the field of interest assigned to the respective virtual agent. The display may be included in a user interface or the user interface may be the display. The display device may be configured to receive generated visual and/or audio signals directly from the processor. The display device may be configured to receive generated visual and/or audio signals based on an emotional state of a user and/or a present behavior and/or a current physical condition and/or the current mental state of the user and to render and cause visualization of each of the at least two virtual agents in order to provide an emotional body language response combined with an emotional oral response to a user.

The ability to mimic another person's actions allows a person to establish a sense of empathy and thus begin to understand another person's emotions. Mirroring can establish rapport with the individual who is being mirrored, as the similarities in nonverbal gestures allow the individual to feel more connected with the person exhibiting the mirrored behavior. As the two individuals in the situation display similar nonverbal gestures, they may believe that they share similar attitudes and ideas as well. Mirror neurons react to and cause these movements, allowing the individuals to feel a greater sense of engagement and belonging within the situation. Mirroring is common in conversation, as the listeners will typically smile or frown along with the speaker, as well as imitate body posture or attitude about the topic. Individuals may be more willing to empathize with and accept people whom they believe hold similar interests and beliefs, and thus mirroring the person with whom one is speaking may establish connections between the individuals involved. Individuals with autism or other social difficulties may be less likely to exhibit mirroring, as they may be less subconsciously and consciously ware of the action of others. This factor may cause additional difficulties for the individuals, as without mirroring, establishing connections with other people may be more difficult. Additionally, other individuals may be less likely to build rapport with the person, as without mirroring the person may seem more dissimilar and less friendly. Individuals who are not subconsciously aware of gesture may have difficulties in social situations, as they may be less able to understand another person's perspective without it being explicitly stated, and thus may not understand covert cues that are often used in the social world. Thus, in one embodiment of the present invention a team of at least two virtual agents configured to provide recommendations to a first user may be further configured to provide recommendation to a first user for a current state of a second user, wherein the current state of the second user is determined on the basis of user-specific parameter of the second user, wherein the at least two virtual agents may be configured to acquire user-specific data of the second user for at least one user-specific parameter of the second user using at least one input device, e.g. a sensor, the user-specific parameter comprising activity data of the second user and/or physio-psychological data of the second user, and wherein the recommendations are provided in response to monitored changes of the acquired user-specific data of the second user for the at least one user-specific parameter of the second user from the activity data of the second user and/or the acquired physio-psychological data of the second user, the recommendations for the at least one determined current state of the second user. The at least two virtual agents may be further configured to provide a visual response to a user through a color change, a color, a posture, and/or a motion of the virtual agent combined with an audio response through a sound, a sound volume, an emphasis, and/or an accent of the visualized virtual agent based on a present behavior and/or a current physical condition and/or a current mental state of the user to mirror the user's behavior.

The present invention further relates to a team of at least two virtual agents and/or a system configured to provide a visual response to a user through a color change, a color, a posture, and/or a motion of the respective virtual agent combined with an audio response through a sound, a sound volume, an emphasis, and/or an accent of the respective virtual agent based on a present behavior and/or a current physical condition and/or a current mental state of the user.

In one preferred embodiment each of the two virtual agents may be in form of a stylized chameleon, wherein the form of the stylized chameleon for each virtual agent may be further based on the assigned field of interest of the respective virtual agent.

The present invention further relates to a system for providing a team of at least two virtual agents, the system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest, wherein each of the at least two virtual agents is configured to provide auto-actively recommendations to the user, wherein each of the at least two virtual agents is configured to provide auto-actively recommendations to the user, wherein the recommendations are further based on activity data of the user and/or of physio-psychological data of the user, wherein the physio-psychological data of the user are further based on a present behavior and/or a current physiological condition and/or a current mental state and/or medical condition of the user, wherein each of the at least two virtual agents is displayed two dimensionally or three dimensionally on a display device, wherein the visual response of each of the at least two virtual agents comprises a posture, and/or a motion of the respective virtual agent and wherein the audio response of each of the at least two virtual agents comprises a sound, a sound volume, an emphasis, and/or an accent of the respective virtual agent on the basis of the activity data of the user and/or physio-psychological of the user, wherein the posture, and/or the motion of each of the at least two virtual agents and wherein the sound, the sound volume, the emphasis and/or the accent of each of the at least two virtual agents is further based on the assigned field of interest of the respective virtual agent, wherein at least one sensor is configured to acquire physio-psychologic parameters of the user directly by speech recognition, face recognition, measurement of pulse, measurement of breathing, measurement of blood pressure, and/or measurement of the electric conductivity of the skin, wherein each virtual agent is in form of a stylized chameleon, wherein the form of the stylized chameleon for each virtual agent is based on the assigned field of interest of the respective virtual agent.

The present invention further relates to a computing device for generating a team of at least two virtual agents, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest, the computing device comprising:
at least one memory for storing computer readable instructions for generating the team of at least two virtual agents
at least one storage device comprising data related to at least two fields of interest, and
at least one storage device comprising user-specific data, and
at least one input device for obtaining user-specific data for one or more user specific-parameter, and
at least one processor for generating the visual and/or audio recommendations of the at least two virtual agents on the basis of a field of interest assigned to the respective virtual agent and on the basis of user-specific data and current situation of a user, and
at least one output device for presenting the generated visual and/or audio recommendations of the at least two virtual agents to the user.

The present invention further relates to a computing device for generating a team of at least two virtual agents, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest, the computing device comprising:
at least one memory for storing computer readable instructions for generating the team of at least two virtual agents
at least one storage device comprising data related to at least two fields of interest, and
at least one storage device comprising user-specific data, and
at least one input device for obtaining user-specific data for one or more user specific-parameter, and
at least one input device for acquiring user-specific data for one or more user specific-parameter for activity data of the user and/or physio-psychologic data of the user by speech recognition, face recognition, measurement of pulse, measurement of breathing, measurement of blood pressure, and/or measurement of the electric conductivity of the skin, and
at least one processor for generating the visual and/or audio recommendations of the at least two virtual agents on the basis of a field of interest assigned to the respective virtual agent and on the basis of user-specific data and current situation of a user, and
at least one output device for presenting the generated visual and/or audio recommendations of the at least two virtual agents

The present invention further relates to a computing device for generating a team of at least two virtual agents, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest, the computing device comprising:
at least one memory for storing computer readable instructions for generating the team of at least two virtual agents
at least one storage device comprising data related to at least two fields of interest, and
at least one storage device comprising user-specific data, and
at least one input device for obtaining user-specific data for one or more user specific-parameter, and
at least one sensor for acquiring directly user-specific data for one or more user specific-parameter for activity data of the user and/or physio-psychologic data of the user by speech recognition, face recognition, measurement of pulse, measurement of breathing, measurement of blood pressure, and/or measurement of the electric conductivity of the skin, and
at least one processor for generating the visual and/or audio recommendations of the at least two virtual agents on the basis of a field of interest assigned to the respective virtual agent and on the basis of user-specific data and current situation of a user, and
at least one output device for presenting the generated visual and/or audio recommendations of the at least two virtual agents

The computing device may comprise an electronic device. Examples of electronic devices may include any portable, mobile, hand-held or miniature consumer electronic device. Further examples of electronic devices are music players, video players, still image players, game players, other media players, music recorders, video recorders, cameras, other media recorders, radios, medical equipment, calculators, cellular phones, other wireless communication devices, personal digital assistances, programmable remote controls, pagers, laptop computers, printers, or combinations thereof. Further examples of miniature electronic devices are watches, rings, necklaces, belts, accessories for belts, headsets, accessories for shoes, virtual reality devices, other wearable electronics, accessories for sporting equipment, accessories for fitness equipment, key chains, or combinations thereof.

Other devices may include a personal computer, a mainframe computer, a laptop computer, a tablet computer, a cell phone, a smartphone, a smartwatch, a personal digital assistant (PDA) and/or an e-reader device.

The present invention further relates to a computer-implemented method for provision of a team of at least two virtual agents running on a system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest. The bidirectional, multidirectional, or even contradictory recommendations are provided in close temporal succession such as within 20 or 40 or 60 seconds.

Furthermore, the present application is directed to a computer-implemented method for stress reduction of a user running on a system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest.

The reduction of stress reduces or eliminates the risk of cardiovascular diseases and disorders such as cardiac arrhythmia, stroke, and heart attack.

Moreover, the present application is directed to a computer-implemented method as described above, wherein the method comprises the following steps:
a) determining the current state of the user by the data obtained from the at least one input device,
b) accessing the user-specific data stored in one or more databases together with the data obtained in step a),
c) selecting at least two fields of interest which are identified as being important for the user in his current state by the assessment of step b),
d) assigning each of the at least two fields of interest to one of the virtual agents,
e) generating on the basis of the assessment of step b) recommendations of the at least two fields of interest, wherein the recommendations are bidirectional, multidirectional, or even contradictory, and
f) providing the bidirectional, multidirectional, or even contradictory recommendations generated in step e) to the user by the at least two virtual agents through the output device in a close temporal succession.

The present invention also relates to a computer-implemented method for generating a team of at least two virtual agents running on a system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest, the method comprising the steps:
a) determining, by the at least one central processing unit, a field of interest for each of the at least two virtual agents, wherein at least two virtual agents are assigned to a different field of interest,
b) assigning, by the at least one central processing unit, a field of interest to each of the at least two virtual agents,
c) accessing, by the at least one central processing unit, user-specific data stored in one or more databases, or
   acquiring and/or obtaining user-specific data and optionally storing said user-specific data in one or more databases
d) accessing, by the at least one central processing unit, data related to the fields of interest stored in one or more databases,
e) determining, by the at least one central processing unit, a current state of the user on the basis of user-specific data
f) generating, by the at least one central processing unit, on the basis of the user-specific data, the current state of the user and the field of interest assigned to the respective virtual agent visual and/or audio recommendations to the user for increase of well-being
g) providing the generated recommendations to the user on an output device

The method may further include one or more of the steps of:
h) acquiring, activity data of the user and/or physio-psychological data of the user over time from at least one sensor,
i) acquiring, by the processor, activity data of the user and/or a physio-psychological of the user from the at least one sensor
j) analyzing, by the processor, the activity data of the user and/or physio-psychological of the user,
k) determining, by the processor, a current state of the user further on the basis of the acquired activity data of the user and/or the acquired physio-psychological data of the user,
l) rendering, by the graphic processing unit, each of the at least two virtual agents on a display device
m) displaying, by at least one display, each of the at least two virtual agents on the display device
n) repeating the steps of acquiring, by the processor, activity data of the user and/or physio-psychological data of the user
o) analyzing, by the processor, the acquired activity data of the user and/or the physio-psychological of the user
p) determining, by the processor, changes of the acquired activity data of the user and/or the acquired physio-psychological of the user,
q) updating, by the processor, the current state of the user
r) rendering, by the graphic processing unit, each of the at least two virtual agents
s) displaying, by the display, each of the at least two virtual agents

Computing systems generally consists of three main parts: the central processing unit (CPU) that processes data, a memory that holds the programs and data to be processed, and I/O (input/output) devices as peripherals that communicate with a user. The present invention further relates to a computing system configured to generate a team of at least two virtual agents running on a system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest.

The system may further comprise one or more hardware processors configured by machine-readable instructions to determine recommendations to a user and further configured to provide recommendations to a user and may be further configured to determine a visual response combined with an audio response of each of the at least two virtual agents to the user, wherein the recommendations and the visual response combined with the audio response may be based on a field of interest assigned to the respective virtual agent and user-specific data and a current state of the user and may be further based on input data relating to activity data of a user and/or physio-psychological data of a user which may be transmitted by at least one input device and/or at least one sensor and/or are based on said transmitted input data of activity data of a user and/or physio-psychological data of a user which may be stored in at least one database and/or at least one memory and/or at least one storage device.

The one or more hardware processors may be further configured to generate visual and/or audio signals to provide a visual response combined with and audio response of each of the at least virtual agents based on the input data of activity data of a user and/or physio-psychological data of a user which may be transmitted by at least one input device and/or at least one sensor and/or based on the present behavior and/or the current physical condition and/or the current mental state of the user, wherein the visual response combined with the audio response of the at least two virtual agents may be given in an emotional oral form combined with an emotional body language reflecting the physical condition and/or the mental state of the user, e.g. the users' emotions and/or mood. The at least two virtual agents may therefore be configured to provide recommendations to a user, wherein the visual response comprises reflecting the physical condition and/or the mental state of the user, e.g. the user's emotions and/or mood. The system may further comprise a display device configured to receive the generated visual and/or audio signals of the generated visual response combined with the generated audio response and to render and cause presentation of the at least two virtual agents on the display to provide the recommendations and thus to provide a visual response combined with an audio response and thus visual and/or audio recommendations to the user. The one or more hardware processors may be further configured to determine a current state (e.g. present behavior, physical condition and/or mental state) of the user based on an analysis of the transmitted input data of activity data of a user and/or physio-psychological data of a user which may be received by the user interface or on the basis of user-specific data of the field of interest or user-specific data in general. The current state may indicate a physiological state of the user, behavior state of the user, psychological state of the user and/or medical state of the user. The user interface may include one or more input devices like one or more sensors configured to generate output signals and configured to transmit input data of activity data of a user and/or physio-psychological data of a user like for example data of verbal and non-verbal behavior of the user. The one or more hardware processors may be further configured to extract specific information and/or specific input data from the output signals generated by one or more input devices and/or one or more sensors. The one or more sensors may include one or more of physiological sensors, audio sensors, and or visual sensors.

A computer is a machine that manipulates data according to a set of instructions called a computer program. The program has an executable form that the computer can use directly to execute the instruction. Because the instructions can be carried out in different types of computers, a single set of source instructions converts to machine instructions according to the central processing unit type. The execution process carries out the instructions in a computer program. The computing system may include a processing unit, a system memory, and a system bus. One or more of the processes described herein may be implemented at least in part as instructions embodied in a non-transitory computer-readable medium and executable by one or more computing devices. In general, a processor receives instructions from a non-transitory computer-readable medium and executes those instructions thereby performing one or more processes including one or more of the processes described herein for providing a team of at least two virtual agents running on a system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user, on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest.

The “system” as disclosed herein comprises at least one central processing unit, at least one non-transitory computer readable storage medium, at least one input device, and at least one output device. The system is configured to display on the at least one output device at least two virtual agents in a way that the user recognizes two visualized virtual agents. The visualized virtual agents could be presented or displayed on or by the output device successively one after another, or simultaneously and the visualized virtual agent communicating with the user could be somehow highlighted or each virtual agent is presented or displayed on or by one output device each. User-specific data and especially health data of the user and user data such as hobbies, habits, preferences and the like provided by the user to the system are stored on the at least one non-transitory computer readable storage medium or in a data bank to which the system has access. User-specific data are not only data directly related to the user like user's health or user's job but also data which are in light of the current state of the user important to the user. Such data could comprise business data of the company the user is working for or business data of a company the user is negotiating with or traffic data of a region the user intends to travel to, heath data of the wife of the user, the curriculum vitae of a person the user intends to meet and the like. The at least one input device, for instance, a camera, sensor, microphone and the like records the current state of the user. The user-specific data and the data of the current state of the user are processed by the at least one central processing unit in a way that the data are analyzed in light of a variety of fields of interest. Thereby each field of interest is selected consecutively and weighted as the most important one so that a recommendation is generated which is in most favor of that most weighted field of interest. In case one field of interest is not important to the processed data, i.e. is not suitable to reduce the stress of the user in his current situation, this field of interest is skipped and the next is processed. After weighting each single field of interest of all the fields of interest as the most important one, at least two recommendations are generated which are bidirectional, multidirectional, or even contradictory to each other. These different or even contrary ways how the user could act in his current situation are presented by the visualized virtual agents in a way that distinctively different from each other each virtual agent presents one recommendation to the user. Consequently, if the system had generated four multidirectional or contradictory recommendations, four visualized virtual agents are generated to present one recommendation each. The bidirectional, multidirectional, or even contradictory recommendations provide a full or almost full analysis of the stress situation to the user, thereby providing to the user a clear picture of the advantages and disadvantages of each recommendation, but leaving the final decision with the user without patronizing and domineering the user. The rational analysis of the stress situation could also give new ideas to the user where the user had not thought about in his stress situation thereby reducing the stress of the user and supporting the user in finding the most suitable way to proceed.

The computing system may further include a bus that transfers data between computer components inside the computing device or between one or more computing devices. The system bus couples system components including, but not limited to, the system memory to the processing unit. The system bus may include several forms of bus structures including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), PCI Express (PCI-e), Card Bus, Universal Serial Bus (USB), Advanced Graphics Port (AGP), Personal Computer Memory Card International Association bus (PCMCIA), Firewire (IEEE 104) and Small Computer Systems Interface (SCSI). The internal bus, internal data bus, memory bus, system bus or Front-Side-Bus connects all the internal components of a computer, such as CPU and memory, to the motherboard. Internal data buses are also referred to as a local bus, because they are intended to connect to local devices. The external bus, or expansion bus, is made up of the electronic pathways that connect the different external devices. Buses can be parallel buses or serial buses. A serial bus can be operated at higher overall data rate than a parallel bus. USB, FireWire, Serial ATA Cache is a small, fast local memory that transparently buffers access to a larger but slower or more distant/higher latent.

The computing system may further comprise a computer-readable data storage that is configured with computer-executable instructions that cause certain functionality to be performed when executed by a processor or central processing unit (CPU). The computer-executable instructions may include a routine, a function, or the like. A component of the computing system may be localized on a single computing device or distributed across several computing devices. The system may comprise a user interface configured to receive input information related to the user like user-specific input data. The system may comprise a user interface, one or more sensors, a display, hardware processor(s), electronic storage, external resources and/or other components. One or more components of the system may be communicatively coupled via a network and/or other coupling mechanisms. Computer-readable storage media may include, but are not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or other tangible and/or non-transitory media which can be used to store desired information. Computer-readable storage media can be accessed by one or more local or remote computing devices, e.g. via access requests, queries or other data retrieval protocols, for a variety of operations with respect to the information stored by the medium. Disk storage includes, but is not limited to, devices like magnetic disk drive, solid state disk (SSD), floppy disk drive, tape drive, Jaz drive, Zip drive, LS-70 drive, flash memory card, memory stick. Disk storage can include storage medium separately or in combination with other storage medium including, but not limited to, an optical disk drive such as a compact disk ROM device, (CD-ROM), CD recordable drive (CD-R Drive), CD rewritable drive (CD-RW Drive) or a digital versatile disk ROM drive (DVD-ROM). To facilitate connection of the disk storage devices to the system bus, a removable or non-removable interface is typically used.

The system may further comprise a memory, such as random access memory (RAM) for temporary storage of information and/or a read only memory (ROM) for permanent storage of information, and a mass storage device, such as a hard drive, diskette, or optical media storage device. The components of the system may be connected to the computer using standard based system, which may include peripheral component interconnect (PCI), Microchannel, SCSI, Industrial standard Architecture (ISA), and extended ISA (EISA) architectures, read only memory (ROM), programmable ROM (PROM), electrically programmable ROM (EEPROM), flash memory. Volatile memory includes random access memory (RAM), which acts as external cache memory. The volatile memory may store the write operation retry logic and the like. RAM is available in many forms such as static RAM (SRAM), dynamic RAM (DRAM), synchronous DRAM (SDRAM) double data rate SDRAM (DDR SDRAM) and enhanced SDRAM (ESDRAM). The system memory may include volatile memory and non-volatile memory. The basic input/output system (BIOS) containing the basic routine to transfer information between elements within the computer, such as during start-up, is stored in non-volatile memory.

The computing system may further comprise at least one processor. The computing system may further comprise at least one processing unit. The one or more processors may be configured to provide information processing capabilities in the system. The processor may comprise one or more of a digital processor, an analog processor, and a digital circuit designed to process information, an analog circuit designed to process information, a state machine, and/or other mechanisms for electronically processing information. Further a single integrated circuit chip and multiples integrated circuit chips. The processing unit can be any of various available processors, also dual microprocessors and other multiprocessor architectures. The at least one processor may comprise a plurality of processing units. These processing units may be physically located within the same device (e.g. a server) or the processor may represent processing functionality of a plurality of devices operating in coordination (e.g. a server, computing devices associated with a user, user interface, medical devices, devices that are part of external resources, and/or other devices). The processor may be configured via machine-readable instructions to execute one or more computer program components. The processor may be configured to execute the one or more components by software, hardware, firmware, some combination of software, hardware, and/or firmware; and/or other mechanism for configuring processing capabilities on the processor. The components may be co-located within a single processing unit. In embodiments in which processor comprises multiple processing units, one or more of the components may be located remotely from the other components. The processor may be configured to execute one or more additional components that may perform some or all of the functionality to one of the components. The computing system may further comprise a central processing unit (CPU), which may comprise a conventional microprocessor. The processor generally represents any type or form of processing unit capable of processing data or interpreting, executing, and/or directing execution of one or more of the instructions, processes and/or operations in accordance with one or more application or other computer-executable instructions such as may be stored in a storage device or another computer-readable medium.

The computing system may comprise one or more input/output devices and interfaces, such as a keyboard, a pointing device like a mouse, a touchpad, a touchscreen, a ring, a printer and the like. The computing system may further comprise one or more display devices, such as a monitor or a touchscreen that allows visual presentation of each of the at least two virtual agents and further may allow visual presentation of data to a user. A display device may provide for the presentation of graphical user interfaces (GUI), application software data, and multimedia presentations. The computing system may further comprise a microphone, motion sensor that allows a user to generate input to the computing system using sounds, voice, motion gestures or the like. The computing system may also comprise input/output devices and interfaces which may provide a communication interface to various external devices via a link to a network. The computing system may also comprise one or more multimedia devices, such as speakers, video cards, graphics accelerator, and microphones. Input devices include, but are not limited to, a pointing device such as a mouse, trackball, stylus, touch pad, keyboard, microphone, joystick, game pad, satellite dish, scanner, TV tuner card, digital camera, digital video camera, web camera, and the like, a receiver, and RF or infrared receiver. These and other input devices connect to the processing unit through the system bus via interface port. Interface port includes a serial port, a parallel port, a game port, and a universal serial bus (USB). Output devices use some of the same type of port as input devices. Thus a USB port may be used to provide input to computer and to output information from computer to an output device. Output devices are devices like monitors, speakers, and printers.

An operating system can be stored on disk storage acts to control and allocate resource of the computer system. Applications take advantage of the management of resources by an operating system through program modules and program data such as the boot/shutdown transaction table and the like, stored either in system memory or on disk storage. It is to be appreciated that virtual agent team can be implemented with various operating systems or combinations of operating systems. The computing system may be controlled and coordinated by operating system software such Windows XP, Windows Vista, Windows 7, Windows 8, Windows 10, Linus, Solaris, Android, iOS; Blackberry OS, Palm OS, Windows Mobile, Windows Phone, or other compatible operating systems. The operating system may control and schedule computer processes for execution, perform memory management, provide file system, networking and input/output services and may provide a user interface such as a GUI and the like.

The user interface may be configured to receive user-specific input data from at least one input device and/or at least one sensor and/or configured to provide visual responses combined with audio responses like visual and/or audio recommendations to one or more users of the system. The user interface may be located in a personal computing device, wearable electronic device, a medical device, and/or locations within or outside the system. The user interface may be configured to provide an interface between the computing system and the user. This enables data, cues, results, and/or instructions and any other communicable items to be communicated between the user, processor, sensors, and/or other components of the system. Visual responses, audio responses, reviews, graphs, predictions and/or other information may be communicated from the system to the user via the user interface. Examples of interface devices suitable for inclusion in a user interface may comprise a graphical user interface, a display, a touchscreen, a keypad, buttons, switches, a keyboard, knobs, levers, speakers, a microphone, an indicator light, an audible alarm, a printer, a haptic feedback device, an optical scanner, a bar-code reader, a camera, and/or other interface devices. The user interface may comprise a plurality of separate interfaces for example a plurality of different interfaces associated with a plurality of computing devices associated with the user. An interface that may be part of a computing device associated with the user, processor, electronic storage, external resources, sensors, and/or other components of the system. A user interface may be included in a server that also includes a processor and/or electronic storage, and/or other interfaces. The user interface may be configured such that a user may receive visual responses combined with audio responses from the system via the individual ones of the plurality of user interfaces. The user interface may comprise at least one interface that is provided integrally with the processor and/or other components of the system.

The system may further comprise a storage component which may include user-specific input data related to the user that is provided by the user, a user of the system and/or provided by other components of the system. The component may be configured to adapt from user-specific input data in real time and dynamically update user-specific input data in the storage component. Other components of the system may be configured to dynamically adjust the analysis and output based on interactions with the user and based on recognized and/or measured physio-psychologic parameters of the user received through means of speech recognition, face recognition, measurement of pulse, measurement of breathing, measurement of blood pressure, and/or measurement of the electric conductivity of the skin in real or near real time.

The system may further comprise a component configured to generate visual and/or audio signals related to visual and/or audio recommendations of the at least two virtual agents. The information related to the at least two virtual agents may include verbal behavioral characteristics and non-verbal characteristics of the virtual agent.

For example, the generated visual and/or audio signals include information about how the virtual agent looks, how it moves, how it reacts to interaction with the user, how it talks, the tone of the voice, the accent, the emotions expressed, and/or other information related to verbal behavioral characteristics and non-verbal characteristics of the at least two virtual agents. The component may include a verbal behavior generator for generating audio responses, a non-verbal behavior generator for generating visual responses, and/or other components. Verbal behavior generator is configured to generate verbal behavior characteristics of the at least two virtual agents, speech recognition including features of speech (e.g. tone, pitch, accent, emotion, etc.), content of speech, and/or other verbal behavior characteristics of the at least two virtual agents. Non-verbal behavior generator may be configured to generate non-verbal behavior characteristics of the at least two virtual agents, for example, appearance of each of the at least two virtual agents, emotional expressions, movements, expressions, body language, posture, and/or other non-verbal behavior characteristics of the at least two virtual agents. Audio and/or visual signals may be provided to the user during the user's performance of activities. Audio and visual signals may include feedback to the user's progress and/or entertainment. The signals may be played at predetermined points during an activity, based on performance metrics, or at the initiation of the user.

The system may include a central server which may include a controller, memory and communication model. A user may download data collected about his activities from the user's electronic device into the central server. The memory of the central server may be configured to store the user's data in a user-specific profile. A local server may include a user's personal computer. User-specific profiles or user-specific input data may be stored in the respective memories of a central server, local server and/or electronic device. The computing system may be coupled to a network such as a LAN, WAN or the Internet, for example, via a wired, wireless or combination of wired and wireless, communication link. The network may communicate with different computing devices and/or other electronic devices via wired or wireless communication links. Access to the electrical system of the computer system by computing systems and/or by data sources may be through web-enabled user access point such as the computing system or data source of personal computer, mobile device, cellular phone, smartphone, smartwatch, laptop, tablet computer, e-reader device, audio player or other device capable of connecting or configured to connect to the network. Such devices may have a browser module or specific application that is implemented as a module that uses text, graphics, audio, video, and other media to present data and to allow interaction with data via the network.

The computing device may operate in a networked environment using logical connections to one or more remote computers, such as a remote computer. The remote computer can be a personal computer, a server, a router, a network PC, a workstation, a microprocessor based appliance, a peer service, a smart phone, a table, or other network node. A Network interface encompasses wire and/or wireless communication networks such as local-area networks (LAN) and wide area networks (WAN) and cellular networks. LAN technologies include Fiber Distributed Data Interface (FDDI), Copper Distributed Data Interface (CDDI), Ethernet, Token Ring. WAN technologies include, but are not limited to, point-to-point links, circuit switching networks like Integrated Services Digital Networks (ISDN) and variations thereof, packet switching networks, and Digital Subscriber Lines (DSL). Hardware and/or Software necessary for connection to the network interface includes, for example, internal and external technologies such as, modems including regular telephone grad modems and DSL modems, ISDN adapters, and wired and wireless Ethernet cards, hubs and routers.

The system may also include one or more servers. The servers can also be hardware or hardware in combination with software. One possible communication between a client and server can be in form of a data packet transmitted between two or more computer processes wherein the data packet may include video data. The data packet can include meta data, like associated contextual information. The system may include a communication framework (like a global communication network such as the Internet, or mobile networks that can be employed to facilitate communication between the client computing devices/electronic devices and the servers Communications can be facilitated via a wired (including optical fiber) and/or wireless technology. The clients may include or are operatively connected to one or more client data stores that can be employed to store information local to the clients (like. associated contextual information). Similarly, the servers are operatively included or are operatively connected to one or more server data store than can be employed to store information local to the servers. A client can transfer an encoded filed to a server. The server can store the file, decode the file, or transmit the file to another client. A client can also transfer an uncompressed file to a server and the server may compress the file. A server may encode video information and transmit the information via communication framework to one or more clients.

Access to the electrical system of the computer system by computing systems and/or by data sources may be through web-enabled user access point such as the computing system or data source of personal computer, mobile device, cellular phone, smartphone, smartwatch, laptop, tablet computer, e-reader device, audio player or other device capable of connecting or configured to connect to the network. Such device may have a browser module or specific application that is implemented as a module that uses text, graphics, audio, video, and other media to present data and to allow interaction with data via the network.

The computer can operate in a networked environment using logical connections to one or more remote computers, such as a remote computer. The remote computer can be a personal computer, a server, a router, a network PC, a workstation, a microprocessor based appliance, a peer service, a smart phone, a table, or other network node. Network interface encompasses wire and/or wireless communication networks such as local-area networks (LAN) and wide area networks (WAN) and cellular networks.

The computing system may comprise a display device like a liquid crystal display, a plasma display or other types and/or combinations of displays. The computing system may comprise a physical or logical connection between a remote microprocessor and a mainframe host computer for the purpose of uploading, downloading, or viewing interactive data and databases online in real time. The remote microprocessor may be operated by an entity operating the computer system including client server systems or main server systems and/or may be operated by one or more of data sources and/or one or more of computing systems. The computing system may communicate with other data sources and/or other computing devices and may comprise one or more internal and/or external data sources. One or more of said data sources may use a relational database as well as other types of databases.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

### Example

The following example is one of million possibilities how the carry out the invention.

The user is a managing director of a small pharmaceutical company which has developed a new pharmaceutical compound for neurological diseases. The small pharmaceutical company has first clinical data and strong patent protection on the new pharmaceutical compound. This had attracted a big pharmaceutical company so that currently the managing director is negotiating with five representatives of the big pharmaceutical company a licensing agreement.

The current situation is that the managing director is together with the five representatives of the big pharmaceutical company in a meeting room at the headquarters of the big pharmaceutical company and the licensing discussions lasted already seven hours. The managing director is quite exhausted and uncertain how to proceed so that during a short break he contacted his virtual advisor team through his mobile phone.

The managing director is wearing a watch which has during the last seven hours taken physiological data like heart rate, blood pressure, temperature, skin conductivity and his mobile phone had recorded during the last 7 hours vocal and verbal data and pictures of his eyes, mouth, and face by the camera in the mobile phone to perform a voice and face recognition. These data were analyzed together with user specific data stored in the system providing the team of virtual agents. Such user specific data comprise, for instance, health data of the managing director and his current health state like suffering from a cold, lack of sleep and the like.

The user specific data stored in the system show that the managing director is not suffering from any chronic disease but recovered from a cold only a couple of days ago.

The user specific data stored in the system and the data obtained through the watch and the mobile phone of the current situation were analyzed by the system in a way that each field of interest like health, ambient situation, nutrition, financial situation etc. is once weighted as the most important one so that recommendations from several perspectives are obtained wherein each recommendation is from the perspective of one field of interest.

In regard to the current situation of the managing director it is obvious that several fields such as hobbies, sexuality and nutrition are for the current situation not of interest while the fields of health and finance are of mayor importance.

Thus, weighting the health field as the most important one the current situation is analyzed by the system as follows. The user is under huge stress over several hours as clearly indicated by his physiological data like heart rate, blood pressure, temperature, skin conductivity. In addition the user just recovered from a cold a couple of days ago as evident from the user specific data stored in the system.

Thus, there is a considerable risk of relapse if the user continues to stress his body like he did during the last seven hours. Consequently the virtual health agent in a visualized form recommends to the user to stop with the negotiations for today and to continue the next day.

Next the second field of importance, the financial field, is weighted as the most important one. From the face recognition, voice recognition, and verbal recognition the system had analyzed that the user has already negotiated several conditions of the license agreement in favor of his company. It is evident that the managing director is doing a good job and succeeding to obtain advantageous licensing conditions for his company. Consequently, the virtual finance agent in a visualized form recommends to continue with the negotiations and to finalize the license deal today, because the user is in a surprisingly good state and good mood to beat the representatives of the big pharmaceutical company, because he has already negotiated licensing conditions very much in favor of his company so that the licensing deal should be closed today not taking the risk that after a break the already negotiated conditions are questioned again tomorrow when the negotiations are continued the next day.

Both recommendations which are contradictory to each other are displayed by the two visualized virtual agents in close temporal succession so that the user, i.e. the managing director is aware of both recommendations at the same time. Now it is up to the user to decide how to proceed and he has a clear picture of the consequences. If he continues he might most probably close a quite advantageous licensing deal for his company which is of big financial benefit to his company, but to finish the negotiations might be such exhaustive that he will relapse and will be ill for a certain time. In case he stops the negotiations for today and continues tomorrow or in a couple of days, he will protect his health and lower the risk of a relapse, but will increase the risk that the licensing conditions negotiated in favor of his company will be questioned again and had to be discussed again and might be changed to conditions in more favor of the big pharmaceutical company. Thus, if the negotiations are postponed, he might lose his favorable position he succeeded to obtain by his successful negotiation today.

1. A system for providing a team of at least two virtual agents, the system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest.

2. A system for stress reduction of a user comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest.

3. The system according to claim 1, wherein the field of interest is selected from a group consisting of health, fitness, nutrition, financial situation, work, job opportunities, job situation, social status, family status, relationship status, education, emotional status, ambient situation, hygienic conditions, availability of medicinal products and medical care, hobbies, travelling, housing conditions, insurances, retirement, social and financial security, mobility, social status, material possessions, property, luxury needs, ethnic, cultural, linguistic and religious identity, sexuality, self-discovery, personal wishes and dreams, legal protection, international security, economic development and/or social progress.

4. The system according to claim 1, wherein each of the at least two virtual agents is configured to provide auto-actively recommendations to the user.

5. The system according to claim 1, wherein the recommendations are further based on activity data of the user and/or of physio-psychological data of the user.

6. The system according to claim 1, wherein the physio-psychological data of the user are based on a present behavior and/or a current physiological condition and/or a current mental state and/or medical condition of the user.

7. The system according to claim 1, wherein each of the at least two virtual agents is displayed two dimensionally or three dimensionally on a display device.

8. The system according to claim 1, wherein the visual response of each of the at least two virtual agents comprises a posture, and/or a motion of the respective virtual agent and wherein the audio response of each of the at least two virtual agents comprises a sound, a sound volume, an emphasis, and/or an accent of the respective virtual agent on the basis of the activity data of the user and/or physio-psychological of the user.

9. The system according to claim 8, wherein the posture, and/or the motion of each of the at least two virtual agents and wherein the sound, the sound volume, the emphasis and/or the accent of each of the at least two virtual agents is based on the assigned field of interest of the respective virtual agent.

10. The system according to claim 1, wherein at least one sensor is configured to acquire physio-psychologic parameters of the user directly by speech recognition, face recognition, measurement of pulse, measurement of breathing, measurement of blood pressure, and/or measurement of the electric conductivity of the skin.

11. The system according to claim 1, wherein each virtual agent is in form of a stylized chameleon, wherein the form of the stylized chameleon for each virtual agent is based on the assigned field of interest of the respective virtual agent.

12. A computer-implemented method for provision of a team of at least two virtual agents running on a system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest.

13. A computer-implemented method for stress reduction of a user running on a system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest.

14. The computer-implemented method according to claim 12, the method comprising the steps:
a) determining the current state of the user by the data obtained from the at least one input device,
b) accessing the user-specific data stored in one or more databases together with the data obtained in step a),
c) selecting at least two fields of interest which are identified as being important for the user in his current state by the assessment of step b),
d) assigning each of the at least two fields of interest to one of the virtual agents,
e) generating on the basis of the assessment of step b) recommendations of the at least two fields of interest, wherein the recommendations are bidirectional, multidirectional, or even contradictory, and
f) providing the bidirectional, multidirectional, or even contradictory recommendations generated in step e) to the user by the at least two virtual agents through the output device in a close temporal succession.

15. The computer-implemented method according to claim 12, wherein the method reduces stress of the user and/or reduces or prevents cardiovascular diseases and disorders, cardiac arrhythmia, stroke, and heart attack.

16. A computing device for provision of a team of at least two virtual agents running on a system comprising at least one central processing unit, at least one non-transitory computer readable storage medium, at least one input device, and at least one output device, wherein the virtual agents are distinctively presented to a user by the at least one output device and each of the at least two virtual agents is assigned to a different field of interest and each of the at least two virtual agents is configured to provide visual and/or audio recommendations of the field of interest assigned to the respective virtual agent to the user on the basis of user-specific data and on the basis of the current state of the user, wherein at least two virtual agents of the team assigned to at least two different fields of interest provide recommendations to the user in the current state so that the user is provided with bidirectional, multidirectional, or even contradictory recommendations of at least two different fields of interest, the computing device comprising:
at least one memory for storing computer readable instructions for generating the team of at least two virtual agents
at least one storage device comprising data related to at least two fields of interest, and
at least one storage device comprising user-specific data, and
at least one input device for obtaining user-specific data for one or more user specific-parameter, and
at least one processor for generating the visual and/or audio recommendations of the at least two virtual agents on the basis of a field of interest assigned to the respective virtual agent and on the basis of user-specific data and current situation of a user, and
at least one output device for presenting the generated visual and/or audio bidirectional, multidirectional, or even contradictory recommendations of the at least two virtual agents to the user.

17. The system according to claim 2, wherein the field of interest is selected from a group consisting of health, fitness, nutrition, financial situation, work, job opportunities, job situation, social status, family status, relationship status, education, emotional status, ambient situation, hygienic conditions, availability of medicinal products and medical care, hobbies, travelling, housing conditions, insurances, retirement, social and financial security, mobility, social status, material possessions, property, luxury needs, ethnic, cultural, linguistic and religious identity, sexuality, self-discovery, personal wishes and dreams, legal protection, international security, economic development and social progress.

18. The system according to claim 2, wherein each of the at least two virtual agents is configured to provide auto-actively recommendations to the user.

19. The system according to claim 2, wherein the recommendations are further based on activity data of the user and/or of physio-psychological data of the user.

20. The system according to claim 2, wherein the physio-psychological data of the user are based on a present behavior and/or a current physiological condition and/or a current mental state and/or medical condition of the user.

21. The system according to claim 2, wherein each of the at least two virtual agents is displayed two dimensionally or three dimensionally on a display device.

22. The system according to claim 2, wherein the visual response of each of the at least two virtual agents comprises a posture, and/or a motion of the respective virtual agent and wherein the audio response of each of the at least two virtual agents comprises a sound, a sound volume, an emphasis, and/or an accent of the respective virtual agent on the basis of the activity data of the user and/or physio-psychological of the user.

23. The system according to claim 22, wherein the posture, and/or the motion of each of the at least two virtual agents and wherein the sound, the sound volume, the emphasis and/or the accent of each of the at least two virtual agents is based on the assigned field of interest of the respective virtual agent.

24. The system according to claim 2, wherein at least one sensor is configured to acquire physio-psychologic parameters of the user directly by speech recognition, face recognition, measurement of pulse, measurement of breathing, measurement of blood pressure, and/or measurement of the electric conductivity of the skin.

25. The system according to claim 2, wherein each virtual agent is in form of a stylized chameleon, wherein the form of the stylized chameleon for each virtual agent is based on the assigned field of interest of the respective virtual agent.

26. The computer-implemented method according to claim 13, the method comprising the steps:
a) determining the current state of the user by the data obtained from the at least one input device,
b) accessing the user-specific data stored in one or more databases together with the data obtained in step a),
c) selecting at least two fields of interest which are identified as being important for the user in his current state by the assessment of step b),
d) assigning each of the at least two fields of interest to one of the virtual agents,
e) generating on the basis of the assessment of step b) recommendations of the at least two fields of interest, wherein the recommendations are bidirectional, multidirectional, or even contradictory, and
f) providing the bidirectional, multidirectional, or even contradictory recommendations generated in step e) to the user by the at least two virtual agents through the output device in a close temporal succession.

27. The computer-implemented method according to claim 13, wherein the method reduces stress of the user and/or reduces or prevents cardiovascular diseases and disorders, cardiac arrhythmia, stroke, and heart attack.
